(19) United States
(12) Patent Application Publication
(10) Pub. No.: US 2020/0187841 A1
(43) Pub. Date: Jun. 18, 2020

(54) System and Method for Measuring Perceptual Experiences

(71) Applicant: Cerebian Inc., Toronto (CA)

(72) Inventor: Karim AYYAD, Kitchener (CA)

(21) Appl. No.: 16/519,301

(22) Filed: Jul. 23, 2019

Related U.S. Application Data
(63) Continuation of application No. PCT/CA2018/050116, filed on Feb. 1, 2018.
(60) Provisional application No. 62/453,022, filed on Feb. 1, 2017.

(51) Int. Cl. A61B 5/16; A61B 5/11; A61B 5/0484; A61B 5/00; G06F 3/01; G06N 3/08; G06N 3/04; G06N 20/20; G06N 20/10; H04L 29/08

(52) U.S. Cl. CPC A61B 5/165; G06F 2203/011; A61B 5/04842; A61B 5/04845; A61B 5/4806; A61B 5/6803; A61B 5/0022; G06F 3/015; G06F 3/017; G06N 3/08; G06N 3/0454; G06N 20/20; G06N 20/10; H04L 67/12; A61B 5/11

(57) ABSTRACT

There is provided a method for determining perceptual experiences. The method comprises obtaining a plurality of signals acquired by a measurement device comprising a plurality of sensors positioned to measure brain activity of users being measured by the measurement device; providing the plurality of signals, without pre-processing, to a processing system comprising at least one deep learning module, the at least one deep learning module being configured to process the signals to generate at least one capability, wherein combinations of one or more of the at least one capability form the perceptual experiences; and providing an output corresponding to a combination of one or more of the at least one capability to an application utilizing the corresponding perceptual experience.

### CROSS-REFERENCE TO RELATED APPLICATION(S)

This application is a continuation of PCT Application No. PCT/CA218/050116 filed on Feb. 1, 2018, which claims the benefit of priority to U.S. Provisional Patent Application No. 62/453,022 filed on Feb. 1, 2017, both incorporated herein by reference.

### TECHNICAL FIELD

The following relates to systems and methods for measuring perceptual experiences.

### BACKGROUND

Interfaces that connect a brain's neurons to an external device are typically referred to as Brain Computer Interface (BCIs) or Brain Machine Interfaces (BMIs). Existing BMI applications are limited in their efficiency and for that reason have therefore not been commercially adopted at scale. These applications are found to be limited mainly due to their pipeline of data collection, analysis, and calibration.

It is an object of the following to address at least one of the above-mentioned disadvantages.

### SUMMARY

The following provides a novel implementation to enable not only global adoption of a core technology for determining perceptual experiences, but also enables capabilities such as reconstructing a user's visual and auditory experiences, brain-to-text, and the recording of dreams to name a few.

In the following there is provided a system and method that enables the determination of perceptual experiences or otherwise to determine human perception. Signals are generated from observations or measurements of brain activity and provided to a system or device component such as an application programming interface (API) for use in one or more capabilities that collectively can be considered the perceptual experience of the user. The one or more capabilities executed by the system or device may then be output to one or more applications that desire to know, or rely on receiving, the user's perception or perceptual experience.

In one aspect, there is a method of a method for determining perceptual experiences, the method comprising: obtaining a plurality of signals acquired by a measurement device comprising a plurality of sensors positioned to measure brain activity of users being measured by the measurement device; providing the plurality of signals, without pre-processing, to a processing system comprising at least one deep learning module, the at least one deep learning module being configured to process the signals to generate at least one capability, wherein combinations of one or more of the at least one capability form the perceptual experiences; and providing an output corresponding to a combination of one or more of the at least one capability to an application utilizing the corresponding perceptual experience.

In another aspect, there is provided a computer readable medium comprising computer executable instructions for performing the method

In yet another aspect, there is provided a processing system for determining perceptual experiences, the system comprising at least one processor and at least one memory, the at least one memory storing computer executable instructions for performing the methods.

### BRIEF DESCRIPTION OF THE DRAWINGS

Embodiments will now be described with reference to the appended drawings wherein:

FIG. 1A is a schematic illustration of a user-worn headset configured to obtain brain signals, initiate an API to generate one or more capabilities, and provide the one or more capabilities to one or more applications, onboard the headset;

FIG. 1B is a schematic illustration of a user-worn headset configured to perform at least one of the functions shown in FIG. 1A onboard the headset, and perform at least one of the functions shown in FIG. 1A at a cloud device;

FIG. 1C is a schematic illustration of a user-worn headset configured to utilize both an edge device and a cloud device to process the signals obtained by the headset;

FIG. 2 is a schematic block diagram illustrating a number of exemplary capabilities and applications that can be implemented using the system shown in FIGS. 1A-1C;

FIG. 3 is a schematic diagram of an example 10-20 electrode placement mapping;

FIG. 4 is a flow diagram illustrating a body movement training process;

FIG. 5 is a flow diagram illustrating deployment of body movements within the API;

FIG. 6 is a diagram illustrating a co-registration prior to source localization;

FIG. 7 is a diagram illustrating a source localization;

FIG. 8 is a flow diagram illustrating a weight replacement calibration;

FIG. 9 is a flow diagram illustrating a weight prediction calibration for vision, auditory, and speech;

FIG. 10 is a flow diagram illustrating an emotion recognition process;

FIG. 11 is a flow diagram illustrating a first tier vision algorithm;

FIG. 12 is a flow diagram illustrating a second tier vision algorithm;

FIG. 13 is a flow diagram illustrating a first tier auditory algorithm;

FIG. 14 is a flow diagram illustrating a second tier auditory algorithm;

FIG. 15 is a flow diagram illustrating execution of mental commands;

FIG. 16 is a flow diagram illustrating a first tier speech algorithm;

FIG. 17 is a flow diagram illustrating a second tier speech algorithm;

FIG. 18 is a flow diagram illustrating a dilated convolution neural network (CNN);

FIG. 19 is an illustration of Plutchik's Wheel of Universal Emotions;

FIG. 20 is an illustration of Ekman's Universal Emotions;

FIG. 21 is a diagram illustrating free motion detection and control; and

FIG. 22 is a diagram illustrating two-way communication paths between a computer and user(s).

### DETAILED DESCRIPTION

The following describes systems and methods that can be implemented to enable measuring a user's perceptual experience. A perceptual experience can mean or be based on, without limitation:

1. What body movements are made by the user;

2. What emotions are experienced by the user;

3. What the user is looking at, imagining, and remembering (reconstructed in generative video form, image form, and by keyword descriptions);

4. What sounds the user hears (reconstructed in generative audio form, and by keyword descriptions);

5. What brain-commands (through intention and/or imagery) the user exhibits to applications; and

6. Brain-to-speech and brain-to-text observations.

The following system provides various advantages over prior systems to-date. For instance, in training only one user is needed to perform the training, compared to approaches that rely on a plurality of users. The source localization described below has also not been utilized in traditional approaches during training. As discussed in greater detail below, the present system uses source localization to motor cortices during training from the single user.

Prior systems also do not specify that signals should come from motor areas of brain, or which area at all. The present system specifies that signals coming from regions other than motor cortices are considered noise for the purposes of body movements. It has been found that localizing signals during training can greatly improve efficiency of the deep learning model.

For the algorithms described herein, whereas prior systems use traditional signal processing steps such as artifact removal (ICA, PCA), low/band-pass filtering, and average data from all users for each gesture/movement, the present system does not require intermediate signal processing, does not use traditional approaches such as ICA, PCA, and filtering, and does not average the data from a plurality. Averaging the signals forces the prior approaches to use a classical machine learning approach or regression. Contrary to traditional approaches, the present system also does not use frequency bands (such as alpha, beta, gamma, delta derived through intermediary signal processing steps such as Fourier Transforms), or a percentage of the frequency bands as the main indicator of a user's body movements or mental commands. Similarly, the present system does not require intermediary analysis of variance (ANOVA), multi-variate analysis of variance (MANOVA), or wavelet transforms during intermediary signal processing. That is, the present system sends raw signals directly to the deep learning module(s), does not use classical machine learning, or use the traditional signal processing techniques. As such, the use of “machine learning” in the presently described system precludes the use of ‘classical’ machine learning algorithms, such as support vector machine, logistic regression, naïve bayes. That is, references herein to use of machine learning by the system is referring to deep models.

It may be noted that references herein to traditionally implemented intermediary signal processing steps refers to fixed methods a priori that transform the signal prior to sending it to the machine learning algorithm (i.e. deep learning). Fixed methods such as ANOVA, MANOVA, signal averaging to find evoked response or event-related potentials (ERP). For example, the present system would not need to isolate frequency bands prior to sending the data to the deep learning process. However, the deep learning algorithm itself may find a shared pattern that resembles that, but it finds that pattern more effectively when the method of doing that is not fixed a priori, like using a fast Fourier transform.

Moreover, specific types of neural networks are used to model distribution of data—such as the ADCCNN (Autoregressive Dilated Convolutional Neural Network).

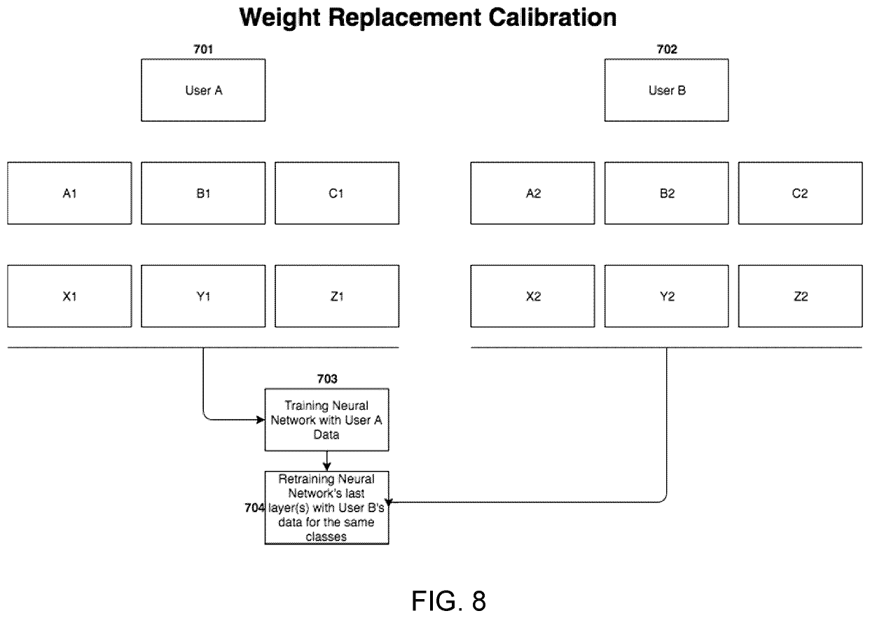

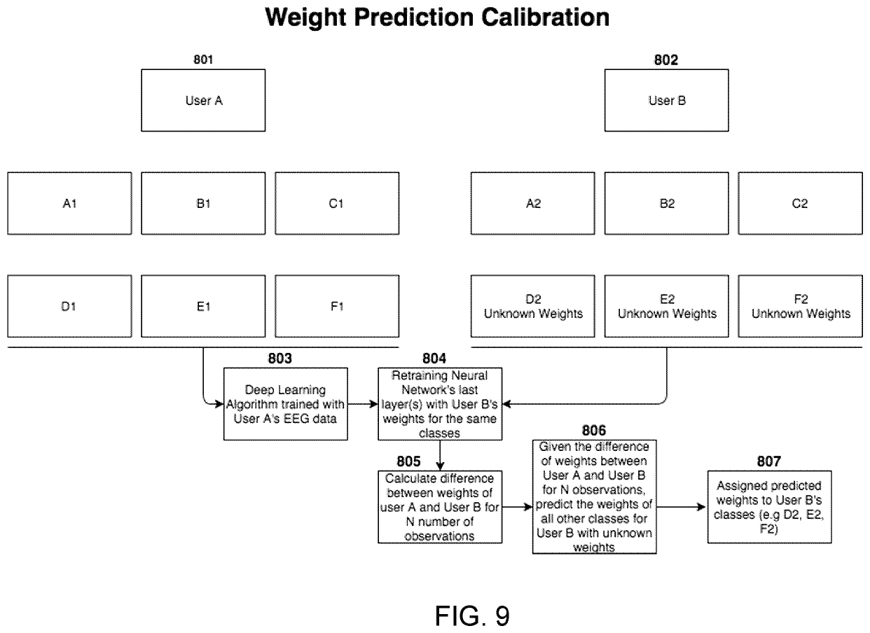

In terms of calibration, using the approach of averaging signals from plurality of users forces the prior approaches to use a generic algorithm generalized for all users. This so-called “calibration” should not be considered a calibration because it forces the user to go through an arduous process of tailoring it specifically for them. In contrast, the present system provides a novel approach for calibrating body movements (see FIG. 8 if only subset of body movements, and FIG. 9 if calibrating for full body modelling/detection). With the present system, every user's model is individualized, with little to no setup. The present system has been found to be less computationally intensive, less arduous, commercially scalable, and importantly, more accurate.

The present system not only enables “continuous motion control”, but goes steps further enabling what is described below as “Free Motion Control”.

These factors and differentiators, combined together, render the whole pipeline of the present system unique to these prior approaches.

Another difference to note is that body movements, unlike traditional approaches, is used in combination with other capabilities described below. Gestures/mental-commands can be used to control a user interface that also adapts according to the user's emotions. Body movements are not only used as gestures to control a UI they're also used to monitor a user's activity.

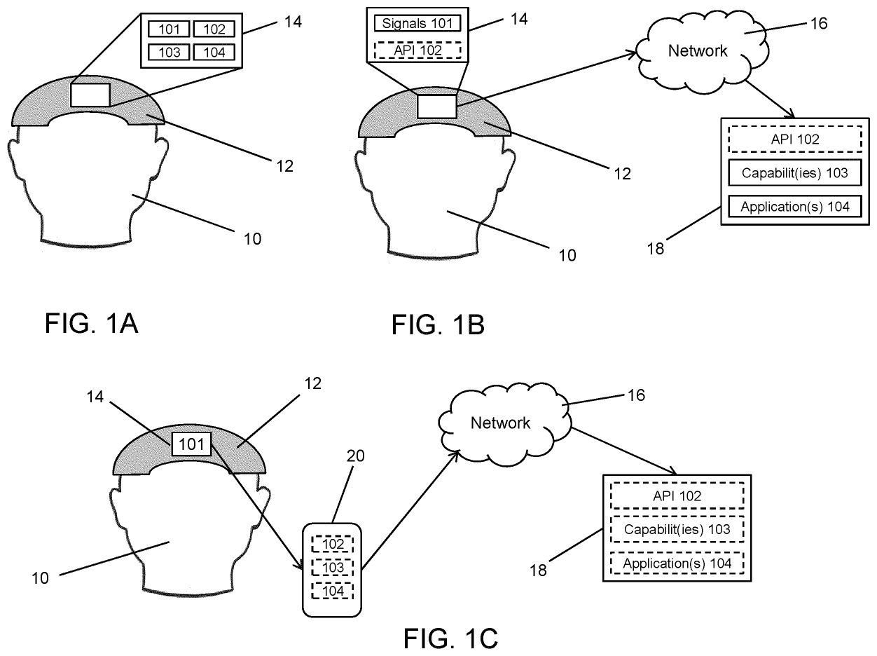

Turning now to the figures, FIGS. 1A to 10 provide exemplary implementations for the system described herein. In FIG. 1A, a user 10 is wearing a headset 12 that includes a plurality of sensors (either non-invasive or invasive), that generate signals 101 corresponding to certain brain activity, such as, without limitation electroencephalography (EEG) signals. In addition to EEG sensors, other types of neuroimaging hardware that is capable of deriving signals that represent brain activity, can be used. For example, blood flow such as fMRI can be measured, whether through ultrasound, implanted electrodes, ECoG, Neural Lace, or other hardware, for example optical imaging methods such as quasi-ballistic photons. As such, it can be appreciated that while certain examples below refer to EEG signals, the principles discussed herein should not be limited to such an implementation.

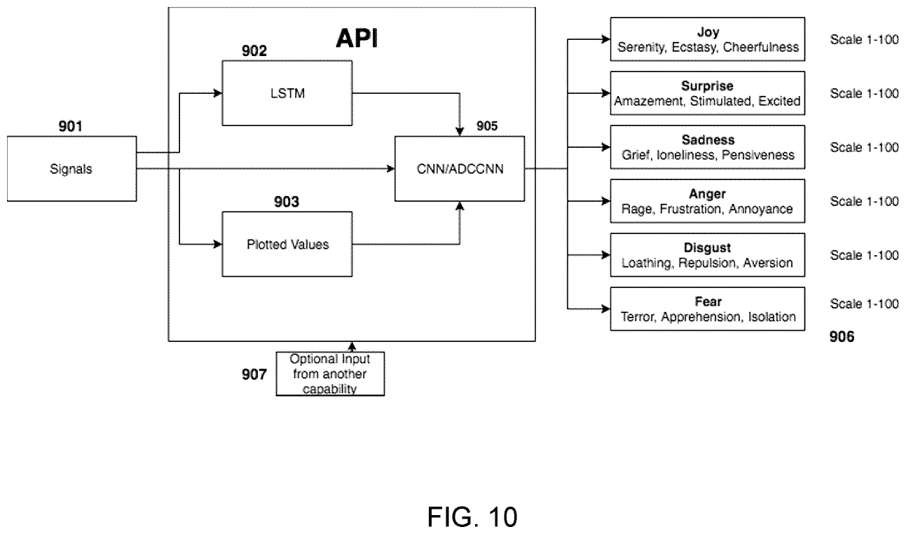

In the implementation in FIG. 1A, the headset 12 includes an onboard module 14 (comprising hardware and/or software) that is configured to acquire the signals, provide the signals to an API 102 (or other device, system, execution module or technology operating in a similar manner) in order to execute, generate, or provide one or more capabilities 103, that is/are fed into one or more applications 104. It can be appreciated that as shown in FIGS. 1B and 10, there are various other possible implementations.

In FIG. 1B, the module 14 is responsible for acquiring the signals 101 and optionally executing the API 102 to provide data over a network 16 to a cloud device 18 (e.g., server or platform) that is configured to execute or implement, one or more of the API 102, the capabilities 103, and the applications 104 at the cloud device 18. In this implementation, the module 14 or the headset 12 comprises a communication interface (not shown) such as a cellular, WiFi or other suitable connection to the network 16.

In FIG. 10, the module 14 is configured to only acquire the signals 101 and send those signals 101 (e.g. in a raw format) to the cloud device 18, via the network 16 and an edge device 20 coupled to the headset 12. As demonstrated by using dashed lines in FIGS. 1B and 10, various configurations can be implemented wherein at least one function is handled onboard the headset 12, with one or more functions performed by the cloud device 18 and/or using an edge device 20. The edge device 20 can be a custom module or a capability added to an existing device such as a smart phone, wearable device, tablet, laptop, gaming device, or any other portable or mobile device. It can be appreciated that in the example configurations shown in FIG. 1, the API infrastructure can utilize distributed computing such as a network of GPUs or block chain based networks.

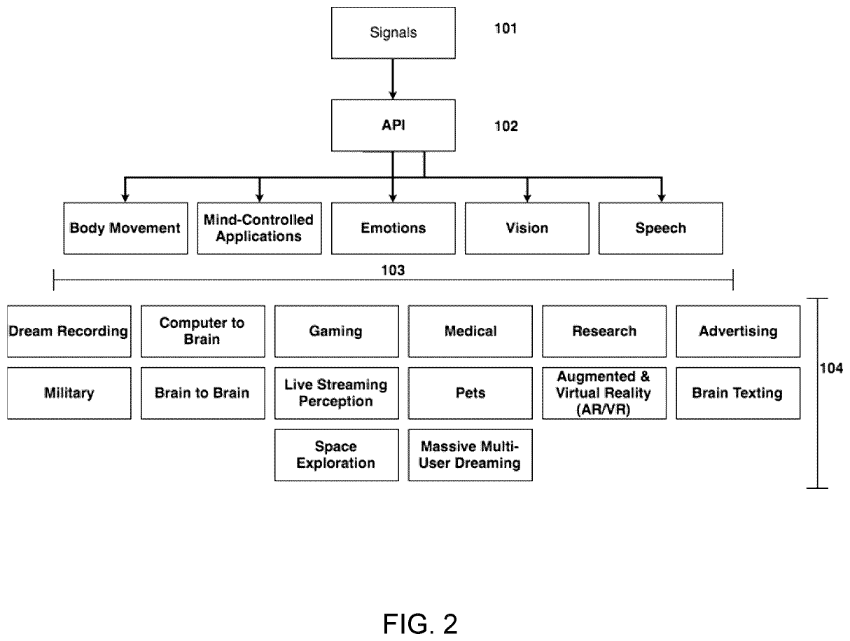

Turning now to FIG. 2, various example capabilities 103 are shown, which together can form a perceptual experience of the user 10. In this example, the API 102 receives a user's EEG signals 101 as an input from an EEG headset 12, and provides as an output one or more (including, for example, every one) of the capabilities 103 for illustrative purposes in the figure. As will be explained in greater detail below, the API 102 or equivalent functionality provides a core technology (i.e. a collection of capabilities 103) that can be used to power many different applications 104, not only the ones listed by way of example in FIG. 2. The applications 104 are therefore listed for the purpose of illustration and to demonstrate what is possible using the disclosed core technology.

In FIG. 2, the signals 101 are in this case generated from an EEG headset 12, and are provided to an API 102. As illustrated in FIGS. 1A-1C, the API 102 can be deployed in an edge-based configuration, e.g., on a mobile device, personal device, computer; and/or using at least some cloud-based software that is trained on receiving a user's EEG signals 101 from the headset 12, decoding the aforementioned capabilities 103 (i.e. the perceptual experience) from a user's brainwaves, and providing the result as an output. The output of the API 102 can be used to power applications 104 in the areas show in the figure, by way of example only.

### Decoding a User's Body Movements

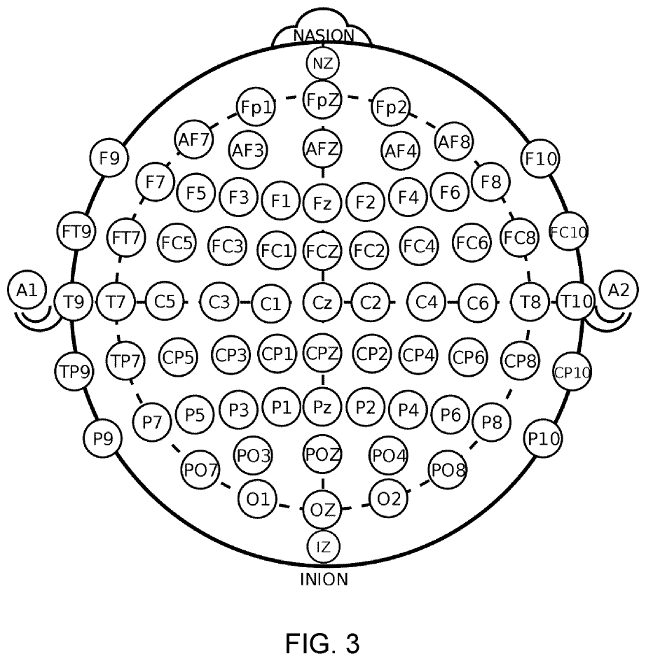

A user's body movements can be decoded by first using scanned signals 101 represented on the motor cortical areas of the brain as an input. Referring now to FIG. 3, an International 10-20 Electrode Placement System is shown by way of example, with the relevant sensors to measuring a user's body movements in this example are:

F7, F5, F3, F1, FZ, FT7, FC5, FC3, FC1, FCZ, T7, C5, C3, C1 and CZ which are on the left side of the brain, used as input to measuring a user's right-side-of-body's movements. For example, moving the right arm, fingers, leg, toes and movement of any and all body parts on the right side.

FZ, F2, F4, F6, F8, FCZ, FC2, FC4, FC6, FC8, CZ, C2, C4, C6, and T8 are sensors on the right side of the brain used as input to measuring a user's left-side-of-body's movements. Such as moving the left arm, fingers, leg, toes and movements of any and all body parts on the left side.

Once the API 102 is deployed in a device or product (or as a product or device), EEG signals 101 received from electrode sensors placed according to the aforementioned 10-20 placement system are then used as input to the API 102 in raw form, with no intermediary signal processing steps. A machine learning algorithm within the API 102 receives the user's EEG signals 102 and a process for building this body movement capability 103 is as follows.

First, the machine learning algorithm is trained on detecting the desired body movements.

### Training:

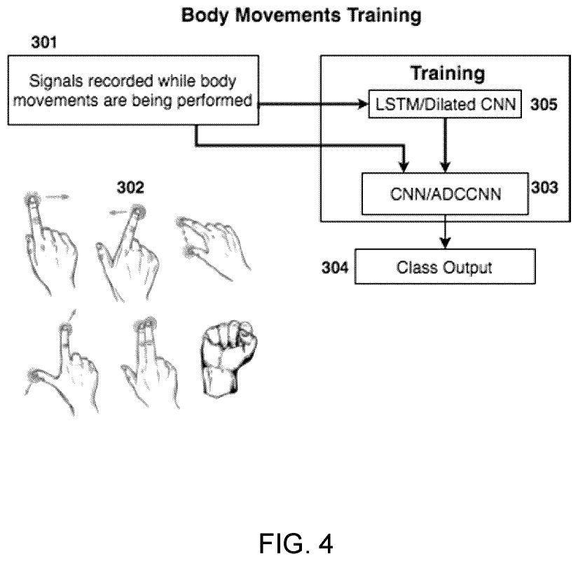

By way of example, in order to detect six different (targeted/pre-determined) body movements, there typically needs to be a data collection (training) session where six different (targeted) body movements are performed by a user in trials while their brain signals 101 are being measured by EEG electrodes as seen in 301 of FIG. 4. An example of body movements is shown in 302. It should be noted that this is not only used for a limited number of human-computer interactions, but goes beyond that. One example is to also measure a user's body movements in terms of monitoring body language and physical activity, which applies to many applications, and the approach extends to measuring each and every body part where possible. The user's generated EEG signals 101 are measured from the aforementioned sensor placements and labelled (with every epoch/period of data corresponding to what body movement was performed during that time of training). The collected dataset is then used to train the machine learning algorithm by way of classification (i.e. deep learning) in block 305 and/or block 303, as will be described below.

### Source Localization:

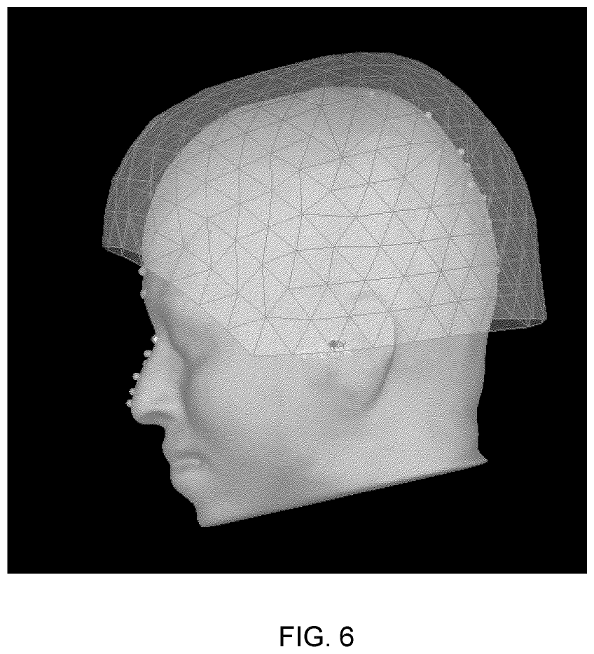

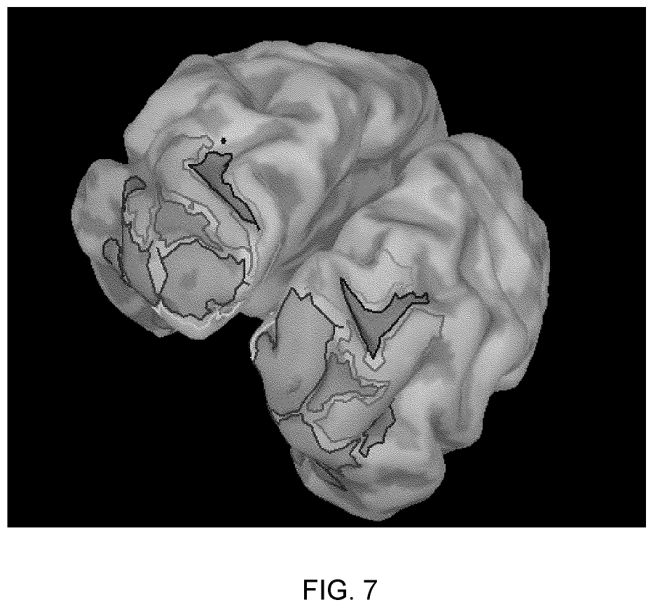

Referring also to FIGS. 6 and 7, in order to collect the most accurate and cleanest data for training the machine learning algorithm that is collected during the training session, source localization can (and is preferable to) be implemented. Not localizing the source of the signals 101 derived from the sensors would not completely fail this approach, nevertheless, it is recommended to derive signals 101 specifically from the targeted areas of the brain to achieve maximum efficiency and accuracy. While traditionally, attempts to construct this capability were made by using all sensors available, data coming from brain regions that are not motor related (and source localized) are considered noise in the present implementation, as it provides features that are irrelevant to the end-result, which renders it less accurate and is considered a reason why this approach has not yet been commercially used on scale.

In order to do source localization, the user whose data is being collected during the training session (by way of example, called User A), undergoes an fMRI scan before the training session starts. A 3D Digitization solution such as the Polhemus-Fastrak as an example, is used in order to digitize points on the user's head. The digitized sensor points are co-registered with the brain anatomy of User A using both their fMRI scan and the output of the digitization solution as can be seen in FIG. 6. Inverse Modelling is employed here and one of a variety of techniques such as LORETA, sLORETA, VARETA, LAURA, Shrinking LORETA FOCUSS (SLF), Backus-Gilbert, ST-MAP, S-MAP, SSLOFO, ALF, as well as beamforming techniques, BESA, subspace techniques like MUSIC and methods derived from it, FINES, simulated annealing and computational intelligence algorithms known to persons skilled in the art of signal processing. A major factor for determining which of the techniques to employ depends on whether there is a fixed number of sensors or not. FIG. 7, by way of example, is localized for visual system, as will be seen in the section on the visual system described below.

Once source localization is completed to desired motor cortical areas of the brain, and the training data is collected, these are provided to the machine learning algorithm for training directly to block 305 and/or block 303 as is described below.

Although traditional machine learning approaches can be used, Convolutional Neural Networks (CNNs) are particularly advantageous for the detection of body movements, and have achieved an accuracy of over 96% in practice. It can be appreciated that more than six body movements can be added by training the deep learning algorithm with more examples of data for different classes (of movements), with the neural network's hyper-parameters and weights optimized accordingly. With more training data, it becomes even more accurate.

Traditionally, EEG signals 101 are filtered using known signal processing techniques like band-pass filtering, low-pass filtering and other techniques such as ICA (Independent Component Analysis), PCA (Principal Component Analysis) which are examples of these techniques. However, the presently described implementation does not employ any of these techniques, while being more effective through this implementation to construct and enable the deep learning algorithm to detect the desired signals 101 rather than resorting to these traditional approaches. Traditional approaches include averaging the signals of each class to find what's known as the Evoked Response (the average signal for a specific class of body movement), or to find Event Related Potentials (ERP) like P300, isolating frequency bands during intermediary signal processing, applying wavelet transformations, and then training an algorithm such as Logistic Regression or other ‘classical machine learning algorithms’.

The present implementation does not average signals (which reduces the amount of data available for training the algorithm, hence requiring data from a plurality of users which due to their different brains would yield a generic system for all users) as a CNN (as well as other deep learning models) requires a large amount of data for training, but rather optimizes the network to find a shared pattern among all raw training examples provided directly to the network as seen in blocks 305 and 303 of FIG. 4.

There are three variants for training blocks 305 and 303. The first variant is training the CNN model in block 303 directly from the raw data.

The second variant is constructing an algorithm that first learns the feature representation of the signals through two (or more) different models within the same algorithm rather than just one model, as can be seen in 305 and 303 of FIG. 4. The first stage is a model that learns the features of EEG data, such as a Long-Short-Term-Memory Network (LSTM), which outputs feature vectors for every labelled epoch of data, and provides that output as an input into the second model. The second model is a CNN that receives the feature vectors from the LSTM or Dilated CNN as input and provides the measured classes (of body movements) as output. As seen in 305, a CNN can be employed in 303 with the first model being a Dilated CNN that learns the features of EEG data over long range temporal dynamics.

The third variant is constructing an Autoregressive Dilated Causal Convolutional Neural Network (ADCCNN) that directly receives signals from 301, and adding an optional “student” module to that will allow it to be faster by more than a thousand times when deployed into production. This will be explained in greater detail in the sections below.

The ADCCNN is trained on providing an output of classes that indicates what body movements were made by the user (which happen simultaneously), and indicates that in a sequential manner. Meaning the ADCCNN for the purposes of this capability 103 takes in a sequence of signals and provides as an output a sequence of samples corresponding to what classes were detected as being performed by the user.

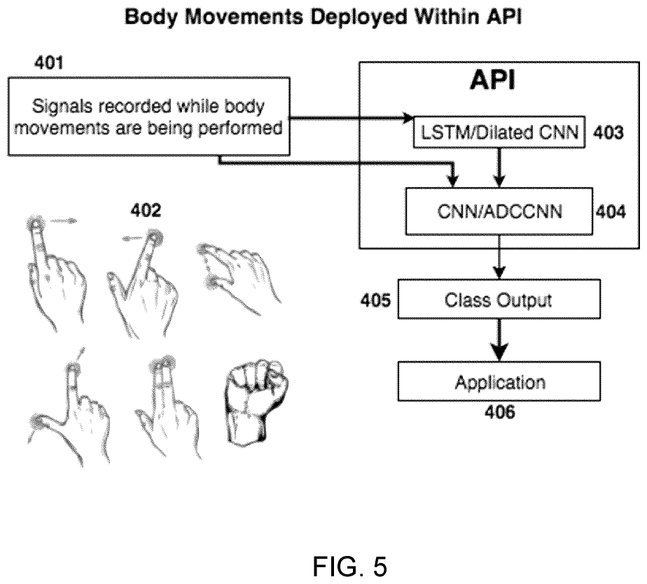

After having trained the algorithm with the defined body movements, the system has a pre-trained model that, along with its optimized weights through training, is deployed within the API 102 for the purposes of decoding body movements through brainwaves as seen in FIG. 5, providing an output in 405 to power any application in 406.

When a new user starts using this API 102, their brain is different due to neuroplasticity, consequently providing different values (degree of variant being dependent on each user) for each class. For this purpose, there is a calibration that is done effectively and in a very short amount of time for any new user of the API.

### Calibration

Turning now to FIG. 8, a weight replacement calibration process is shown.

The deployed pre-trained deep learning model has learned the features of the EEG data in 703. More specifically, every layer of the network as the system goes ‘deeper’, meaning to the next layer of the neural network, learns features of the signal that are less abstract and more specific to the brain of the training dataset's user 10. By way of example, the training dataset here was collected from User A's brain, and User B is a person who will use this technology for the first time. And also by way of example, the network is trained on six body movements performed by User A.

Then, User B wearing the EEG headset 12 is asked through an interface connected to the API 102, to perform again the six classes of body movements. This overcomes the problem of different brains because there is a vast difference between the training process of User A, and the calibration process of User B. First being that training of the neural network for the first time by User A is very extensive and time consuming, and should be done in a controlled environment such as a lab, while User A only moves his body to perform the movements of training classes, with the rest of his body being still. User B's calibration is done in a short amount of time (e.g. 15 seconds in the case of six classes), depending on the number of classes s/he is asked to perform.

Calibration can be done in a commercial setting where the user can be anywhere, rather than a controlled environment. It is also significantly less computationally intensive. While training a deep learning network takes days on a normal CPU (Central Processing Unit), or can be trained with a few hours, minutes, or seconds using GPU (Graphical Processing Unit) depending on how many GPU's are utilized for training, it still requires a very intensive computational power to bring the training time down to seconds or less. This approach's requisites are that User B calibrates with a much smaller dataset than was used during training of User A. For example, five samples for each class was found to be more than enough for the mentioned CNN to calibrate for User B, while achieving near-perfect accuracy.

The calibration process is done by using the same pre-trained deep learning model with the weights optimized to data derived from User A, but removing the last (final layer) of the network, and replacing it with a new layer re-optimized with weights to User B's signals—see 704 in FIG. 8. Through this ‘Transfer of Learning’ approach, User B can start using the technology with only a few examples of training, in a very short amount of time, in a commercial setting, and in a computationally efficient manner.

It may be noted that the deeper the network is (greater the number of layers), the more likely that the system would need to re-optimize the last two layers or more because as mentioned above the more layers go deep, the more they become specific to the data of User A used for initial training. In the CNN mentioned above, remove only the last layer was more efficient than removing the last two.

It may also be noted that due to neuroplasticity, User B's brain is expected to change over time. Hence, ideally the calibration is advised to be done weekly or bi-weekly in a very short amount of time to ensure that maximum accuracy is continually achieved. There is no ideal rate for how often calibration should be done as the neuroplasticity rate is different for each user depending on age and a number of other factors.

While traditionally any attempt to model a user's body movements from their brain signals to power an application 104, has been positioned in a way that when a new user starts using it, it starts learning specifically to their brain from scratch or from a generic baseline, the description here describes two novel calibration methods in FIGS. 8 and 9, and described above which provide many advantages such as calibrating in a short amount of time, being minimally intensive in terms of computation, enables calibration in a commercial setting by any user, and the algorithm does not start learning from scratch, meaning it requires much fewer training examples to calibrate, while maintaining a very high level of accuracy.

Once the API 102 is calibrated to the new User's brain, it will detect a user's body movements with maximum accuracy which can be used to power many applications (see element 406 in FIG. 5) in combination with other capabilities 103 as will be described below.

The results of this capability 103 can be used as input into any one of the other capabilities 103, and/or in combination with them for an application 104.

Traditionally, EEG signals 101 are filtered using fixed preprocessing techniques such as filtering (low-pass, band-pass) to cancel out artifacts and using of techniques like ICA or PCA in order to pre-process the signal before training a machine learning algorithm on it. During that pre-processing, the signals 101 for each given class/motion of body movement are averaged to find the average response also known as evoked response, event-related potentials (ERP) or other traditional signal processing such as P300, which is then used to train a ‘classical machine learning’ algorithm such as logistic regression or a statistical correlator.

This forces the implementer to resort to such classical machine learning algorithms because the use of deep learning algorithms requires a large amount of data. Averaging the signals of a training user, for example if the user did a specific motion during training 100 times, would result in one signal that is the average of all 100 times that represents this motion.

Consequently, the implementer needs to generate data from a plurality of users for every given motion in order to average the signal of all users for a given motion. This is done in order to enable the classical machine learning algorithm to generalize to more than one user, using the average-response of signals generated from the plurality of users for every given motion, and a traditional classical regressor or correlator to do the matching. This creates a generic model baseline for all users that is considered to be much less accurate than the implementation used by the present system. If the user wants a more accurate class/motion detection, then they need to re-do the training steps and use their own neurological data over many trials, which can be very cumbersome to redo and is ineffective, particularly for providing a commercial scalability.

The present implementation does not employ any of the traditionally used techniques mentioned. It is found to be more effective to use deep learning to find a common shared pattern among the signals for a given class/motion with no intermediary signal processing steps. By way of example, if a user during training performs a specific motion 100 times, the signal is not averaged, rather 100 trials of that motion are given to the deep learning algorithm as input. This means that this approach does not need a plurality of users for training, which means it is less cumbersome, less costly, more time-efficient and a lot more accurate (especially when implementing the novel calibration in FIGS. 8 and 9) when deployed into a commercial setting.

The present implementation does source localization as part of the training process specific to the motor cortical areas of the brain, which is not traditionally done, and only one training user is needed to collect the data necessary for the implementation. Rather than having to resort to a generic detector with low accuracy, or a very cumbersome individualized calibration of having to retrain for all classes/motions, the present implementation uses a novel calibration approach discussed herein. Where if the user is calibrating to low number of classes/motions, then weight replacement calibration is done, and if the user wants to calibrate to a fully body modelling, of all classes/motions weight prediction calibration is done, as described herein.

Additionally, the present implementation not only enables the detection of a user's continuous motions (which is considered a natural requirement for the modelling of body movements), but also enables what is termed here as “Free Motion Control”. This notion of free motion control, previously undone before, enables the modelling of a user's body movement in all degrees of freedom, in any degree.

Every motion is by nature continuous. The detection of that is the produced sequence in which models the sequence of motion and speed for each class/motion in block 406. Nevertheless, traditionally motions are detected/classified as, for example, being up, down, right, left, and how long the sequence is detected resembles the degree/level (degree here, meaning the extent—is used with a different meaning than degrees of freedom mentioned above and below) to which a person moved their arm to the right, or extended their foot forward. If a user moves their hand diagonally, traditionally the detection would be for example, Upper Left, Upper Right, Lower Left or Lower Right. Therefore, that is the detection of continuous motion but is not free motion.

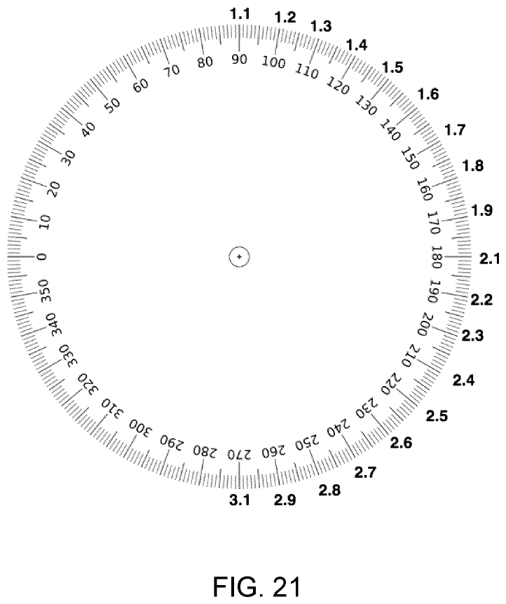

This implementation, as seen in FIG. 21 enables exact modelling of the body-parts motion in terms of its position, and is not limited certain degrees of freedom. Therefore, this not only enables continuous, but also free motion detection and control. Where the output of block 406 models FIG. 21, which is used as an example to show it models exact body movement of the user. The sequential output of block 406 in length determines continuous motion, exact modelling of movement and speed.

For example, a user moving their hand diagonally at a 100-degrees angle, the output of block 406 would be three dimensional value for every epoch/period of time. The output detected by the API 102 in FIG. 5 would be 1.1.5—the first value resembling general direction (Up), the second value resembling exact degree of direction (10 degrees to the right from Up), and the third value resembling the speed of movement. The sequential nature of the output, meaning every epoch after the other, resembles the continuous (and readily apparent) nature of the movement. Once the user's hand stops moving, the directional value of the output zeroes back to a pre-define value resembling that there is no motion in any direction. This enables free motion detection and control, that is not only more advanced than traditional approaches, but is essential for enabling full free control of a prosthetic arm, as an example.

### Decoding a User's Emotions

The decoding of emotions from a user's EEG signals 101 using the API 102 will now be described. This capability 103 enables the API 102 to detect a user's emotions. The first step is to categorize which emotions are to be detected. Emotions are categorized in a number of approaches:

The first variant is what is known as Ekman's Six Universal Emotions: Happiness, Sadness, Surprise, Disgust, Fear, and Anger. These emotions are Universal. Ekman's Emotions are categorized in FIG. 20.

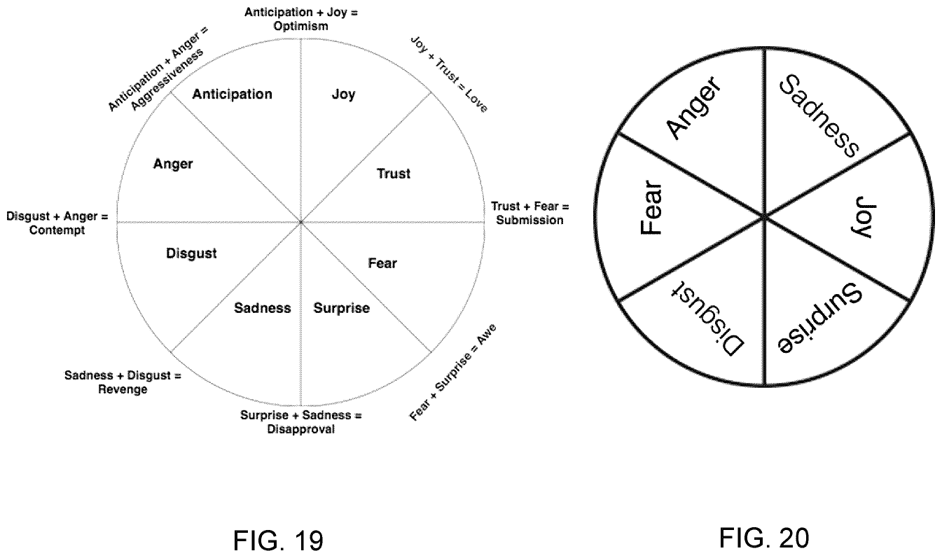

Second categorization of emotions is Plutchik's wheel (see in FIG. 19), which are variants of the same six universal emotions and also include Trust and Anticipation—totaling 8 universal emotions.

The third variant includes and enhances or expands upon the first two variants, to also include any other targeted application specific emotions and mental states, such as motivation, and level of attention. The present system is capable of detecting complex emotions, which is a combination of universal emotions, a capability not implemented in prior approaches. It can be appreciated that combinations of emotions can also yield newer insights.

Generated signals are derived from all available EEG sensors streaming data to the API 102 as seen in 901 in FIG. 10.

By way of example, the first variant which is Ekman's Six Basic Emotions is chosen to provide an example of how an API 102 that automatically detects these emotions is built, trained and deployed.

For the purposes of collecting the training dataset, the name User A will be given to the user that is present during training and undergoes the data collection session.

User A's EEG signals 101 are measured from all sensors available whilst expressing emotions, and that data is labelled with the target expected elicited emotions.

Emotions can be elicited in a number of ways. By way of example a first method is to ask the user to write down a memory associated with an emotion. For example, asking the user during training to write down a happy memory, and collecting a training dataset as the emotions are elicited. Subjective input of the user is taken into account due to subjective nature of emotions to every person. A second method by way of example is to present the training user with audio/video and receiving their subjective input on the type of elicited emotion, and how they grade the level of elicited emotion from 1-10 and using that as an indicator for training the deep neural network more effectively. Therefore, example methods of categorizing target emotions are described, example methods of eliciting emotions are described, and example methods of grading the elicited emotions are described.

After target emotions are defined (in this example, Ekman's Emotions), emotions are elicited, e.g., by asking User A to write down an emotional memory while their signals are measured asking them to grade their emotions subjectively, and by experiencing Audio-Visual and grading their emotional response subjectively as well. The data collected from the EEG sensors are labelled with the expected (objective) and experienced (subjective) input by user.

Data is split into periods of time also known as epochs of data, corresponding to labelled trials of every elicited emotion. Labelled data is then provided to the deep learning algorithm for training in order to classify categorized emotional states in the future. There are no intermediary signal processing steps such as evoked response, ANOVA, MANOVA, wavelet, FFT or other transforms, and frequency bands are not isolated in a fixed manner a priori to train the algorithm. The algorithm directly takes raw data, is trained through deep learning, and includes a number of models.

By way of example, four novel approaches will be provided to construct and train a deep learning algorithm on recognizing user emotions and mental states for any of the three variants of categories described above.

Firstly, a deep learning algorithm is constructed and trained using the following process:

The Algorithm to decode emotions used here is composed of two deep learning models. The first model is an LSTM in 902, which is a type of Recurrent Neural Network (RNN), and is used here as the first model which takes in the raw EEG signals 101, learns their features, and provides them as an output of a feature vector which is used as input to the second model.

The second model used here is a CNN at block 905, which takes as input the feature vectors provided by the first model and further trained in the manner of classification (deep learning) to accurately detect what emotion the user was experiencing through their EEG signals 101.

The deep learning algorithm is not limited to these two types of models, but advantageously or preferably adopts these two models: the first being an RNN which is ideal in picking up and learning the features of EEG over a period of time, as the network has an internal ‘memory’ which uses past data in short-term memory over a long period of time as an input to more efficiently train the network and produce results); the second being a CNN picking up blocks of feature vectors provided by the LSTM. Once this algorithm is trained, it is considered a ‘pre-trained’ algorithm. The algorithm is trained in detecting every one of the emotions independently out of a scale of 1-100, as the user can experience more than one emotion simultaneously.

A second approach to train a deep learning algorithm on the dataset collected from User A can include the following.

The first model is to construct an LSTM that is specific to every channel of EEG available 902. The difference here from the first approach in terms of representing features is that an LSTM is used for every channel. Consequently, if there are twenty eight channels streaming data, then there are twenty eight LSTM Models, that each take a channel's raw data, and output a feature vector for that channel, as opposed to the first approach of a shared LSTM for all channels.

The features of every channel are then passed onto the second part of the algorithm which is a CNN model at 905, which receives the feature vectors provided by every channel and outputs a classification for every chosen emotion using a scale of 1-100.

A third example approach of constructing and training a deep learning algorithm on recognizing emotions can include the following.

EEG data derived from sensors 101 can be fed into an algorithm having two tiers of learning models. The first tier in and of itself comprises two models—one that plots a user's signals in block 903, and an LSTM Model in block 902 that outputs vectors of represented features from the EEG channels (every channel or all channels).

The second tier is a CNN model at 905 that receives two types of inputs—images of the plotted values of every epoch, and LSTM-produced feature vectors of every epoch. The CNN is trained with inputs from the first tier with its hyper-parameters and weights optimized accordingly.

Using a CNN that is pre-trained on images, and removing its last 4 layers (more or less depending on how deep the network is), and then retraining that model with the plotted images of the values, and feature vectors of every epoch, has been found to be more effective and can shortcut the need for more training data.

The fourth approach is to construct an Autoregressive Dilated Causal Convolutional Neural Network (ADCCNN) that will either take signals directly from 901 to 905, or first have the features of the signals learned by the LSTM in 902, and take the output of feature vector that is provided by 902 as an input to the ADCCNN in 905. The ADCCNN will be further explained in detail below. An additional student module can be added to the ADCCNN for advantages explained further below. This approach also does not employ any of the fixed intermediary signal processing steps mentioned above, and signals are sent directly to the deep learning process/module.

An algorithm that was trained using the first, second, third or fourth approach of training is then considered to be a trained algorithm.

The trained algorithm is deployed within the API 102 for the purposes of detecting a user's emotions as seen in 906. Using the first training approach described above, the algorithm has been found in practice to be over 98% accurate, and can be further improved by further optimizing the parameters and weights of the network (with more training examples), or by adding a third modality to the model of the algorithm.

When the API 102 is used to detect emotions of a new user will now be explained.

By way of example, User A was the user whose data was collected to train the algorithm, and User B is a new user. User B is presented with the same stimuli that was presented to User A during training to ensure an effective calibration. The same categorization and grading method is also used. The deep learning algorithm is calibrated to User B through the calibration ‘Transfer of Learning’ process described above—Weight Replacement Calibration as seen in FIG. 8, by positioning the trained algorithm of emotions in 703 and using User B's input to replace the weights using 704.

Weights of the algorithm are then replaced using the weight replacement process. This enables the API 102 to receive EEG signals 101 from a user's brain through the sensors they are wearing and accurately provide an output as to which emotions the user was feeling out of a scale of 1 to 100. By way of example, the user can be 80/100 Angry, and 40/100 Sad, or 100/100 Happy and 60/100 Surprised. Importantly, another approach that has not been known to be done before is combining the categorized emotions to derive measurements of new emotions. As an example, in FIG. 19 a user feeling both fear and trust can suggest a feeling of submission, a user feeling fear and surprise can suggest a user is in awe, a user feeling surprise and sadness can suggest the user disapproves, etc.

An additional third modality can be implemented to receive the output generated by block 906 (see FIG. 10) as to what combinations of categorized universal emotions the user is feeling, and use that data to derive insight into more complex emotions the user is feeling.

The detection of emotions through EEG can also be combined with facial emotions recognition, heart rate, galvanic skin response (GSR), or any other separate modality that will assist the API 102 in more accurately providing the user's emotional response to stimuli.

This capability 103, after being deployed within the API 102, can be used in combination with other capabilities 103 for various applications 104 as will be described below.

The results of this capability 103 can be used as input into any one of the other capabilities 103, and/or in combination with them for an application 104.

### Decoding and Reconstructing a User's Vision

With respect to reconstructing vision, EEG signals 101 are derived from sensors placed on the parietal and occipital areas of the brain, including but not limited to:

P7, P5, P3, P1, Pz, P2, P4, P6, P8, PO7, PO3, POZ, PO4, PO8, O1, OZ, O2 shown in FIG. 3.

Additionally, input can also be derived from the parietal lobe, the inferior temporal cortex and the prefrontal cortex which is involved in object categorization. It can be appreciated that additional sensors can be added, where necessary, to the headset 12, to acquire signals indicative of brain activity, e.g., for the inferior temporal cortex and prefrontal cortex.

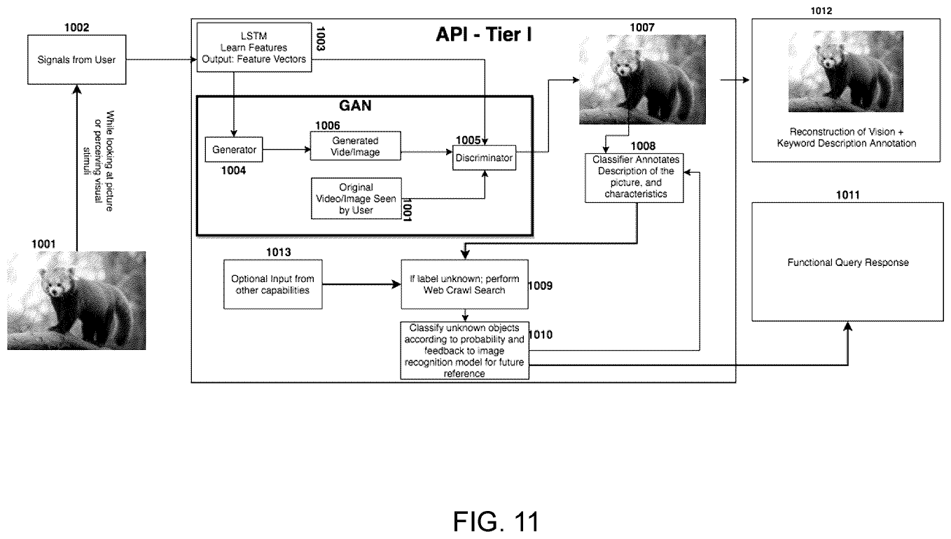

In order to decode in words or keywords what a user is looking at, EEG signals 101 can be measured for User A as seen in 1002 of FIG. 11 (the user whose data is used for collecting the datasets during training) in order to train a deep learning algorithm.

User A undergoes an fMRI Scan, has their head points digitized using a solution such as Polhemus-Fastrak. The fMRI Scan and digitization are both co-Registered as seen in FIG. 6, and the source of the EEG signals 101 are localized from the sensors above to the following areas:

V1-V4, the Fusiform Face Area (FFA), Lateral Occipital Cortex (LOC), Parahippocampal Place Area (PCA), the Lower and Higher Visual Cortices covering areas listed above—the entire visual cortex—as is seen in FIG. 7.

User A looks at image examples of target image categories such as 1001 in FIG. 11. EEG signals 101 are derived from the sensors according to the aforementioned sensor placement for vision and stored as raw EEG signals 101 hereby referred to as ‘vision training data’, that is labelled and split accordingly.

A machine learning (e.g., deep learning algorithm) is constructed and is trained for classification of the vision training data. It has been found that RNNs are ideal networks to learn the features of time-series EEG data. This approach is not limited to an LSTM, however, by way of example, an RNN can be used as seen in block 1003. An LSTM has been found in practice to achieve 97% accuracy, and can be improved further by adding more data, more layers and optimizing the hyper-parameters of the network and its weights accordingly.

The deep learning model, once trained on EEG features of raw data in response to stimuli of images to any specific category of images, can accurately classify a previously unseen image by the user belonging to that same category.

The deep learning model is then deployed within the API 102 along with its weights, ready to receive data and provide as an output what classes of images the user was looking at in keyword descriptions, as is detected from their EEG signals 101.

A calibration as described above is typically required to calibrate from vision training data collected by a training user, to a new user in a new setting (commercial setting for example) as seen in FIG. 9. By way of example, User A at block 801 is the training User, and User B at block 802 is a new user of the technology. User B is presented with images, and the difference in weights between User A's response to image ‘A’ and User B's response to that same image ‘A’ is calculated, and this process is done for a number of images. The difference in weights for every image is used to retrain the deep learning model's last layer (or more depending on depth of the model) through the transfer of learning method described above.

For example—if the model is trained on recognizing one hundred objects seen by User A. When User B starts using the API, they are presented with, by way of example, images of five objects. Objects A1, A2, A3, A4, and A5.

The weight of each class as was trained by User A is X1 for A1, X2 for A2, X3 for A3, X4 for A4, and X5 for Image A5.

When User B is presented with images of the same five objects A1, A2, A3, A4, and A5, the last layer (or more) of the network are retrained for User B. The weights for User B are Y1 for A1, Y2 for A2, Y3 for A3, Y4 for A4, and Y5 for A5. Then Weight Prediction is posed to be: Calculate the difference between Y1 and X1 for Image A1, Y2 and X2 for Image A2, Y3 and X3 for Image A3, Y4 and X4 for Image A4, and Y5 and X5 for Image A5 (see block 805).

Given the difference between X and Y for every image A, predict the weights for all other classes Y6 to Y100 for images A6 to A100, given the known values of X6 to X100, weight prediction calibration is implemented (see block 806).

This calibration approach can enable the deep learning model to adapt to a new user's brain effectively, in a short amount of time, with minimal computational intensity, being viable to be used by a new user in a commercial setting (see block 807).

With regards to generating a video or an image representation of what a user was looking at from their EEG signals 101, this is the reverse process of the visual system of the brain. Light which travels to a person's eye enabling them to see is transformed to electrical signals represented on the cortical areas of the brain. This process hereby uses the electrical signals represented on the cortical areas of the brain to generate the image the person was looking at, or video, through the person's eyes.

In order to generate in video form (or in images) what the user was looking at from EEG signals 101, this can be implemented in two variable approaches for training.

In a first variant, the training User A looks at images belonging to specific categories and their data is used as raw signals to train a neural network to generate the image from EEG signals 101 (see block 1001 in FIG. 11).

In a second variant, the training User A looks at images of shapes and colors, their variants and abstractions, and the building blocks of drawings, effectively collecting data to train the neural networks to draw and generate shapes (and abstract shapes, including colors) from the User A's EEG data.

An algorithm that can be one of two tiers is constructed (with each of the tiers also having the third model and fourth additional modality).

In the first tier as seen in FIG. 11, a deep learning algorithm having three models and a fourth additional modality (with optionally more) is constructed.

The first model is a network that learns and outputs vectors that represent features of EEG data of the raw training data provided. As such, learns the features of EEG data for training User A when they are looking at shapes, colors, and their abstractions. A recurrent neural network, in this case an LSTM at block 1003 is found to be ideal, nevertheless that is not a limitation to what type of network can be deployed here to learn features. A second model is constructed that receives the output of the first model and generates an image or a video using those features, that is as close as possible (and after extensive training becomes exact) to the original training images viewed by the training user (in the first variant), and when deployed it can re-draw (regenerate) images that were not seen during training, when the neural network is trained through the second training variant.

The second model of the algorithm can be a Variational Auto-Encoder (VAE), Convolutional Auto-Encoders, or a Generative Adversarial Network (GAN), Deconvolutional Generative Adversarial Networks, Autoregressive Models, Stacked GAN, GAWNN, GAN-INT-CLAS, or a variant of any of the above to generate an output from the input features of the first model. Where in this case (a GAN), the feature output of the first model of the network (LSTM) is used as input to the two sides of a GAN—the discriminator at block 1005 and the generator in block 1004. Where the generator generates images in block 1006 and the discriminator assesses how accurate the generated image/video is relative to what it should be from the image at block 1001, and provides a feedback loop for the generative portion of the network to improve while the network is being trained.

Once deployed, the second model generates in video form (or image form) exactly what the user was looking at when their EEG data was being recorded, as they were perceiving the visual stimuli as can be seen in blocks 1007 and 1012. Training through the second variant overcomes the traditionally known “open problem of vision”, which states that there is an unlimited amount of objects in the world (as they keep increasing) and that it would not be possible to categorize them all. This overcomes the problem by enabling the network to generate any image or video without having been specifically trained on recognizing that object in the first variant of training. The problem is also overcome in terms of categorizing objects, and not only drawing them, through the feedback loop between blocks 1010-1008, and blocks 1110-1108.

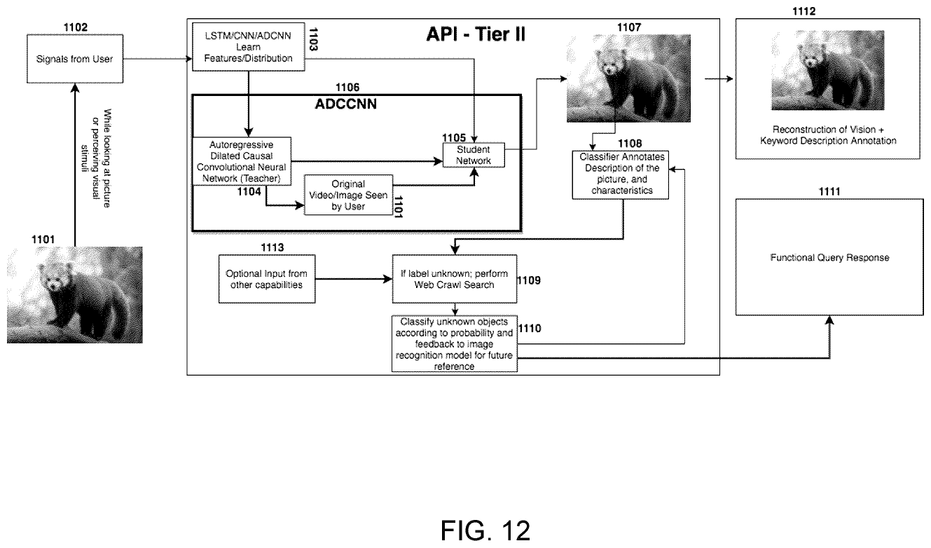

The second tier shown in FIG. 12 of implementing image/video generation can be implemented as follows.

First, the system constructs a unique model in the field of BCIs. The model is based on an ADCCNN applied at block 1106, which exhibit very large receptive fields to deal with the long ranged temporal dynamics of input data needed to model the distribution of, and generate pixels from, the brain-signals. The ADCCNN takes input directly from block 1102.

Each sample within an epoch/period of data is conditioned by the samples of all previous timestamps in that epoch and epochs before it. The convolutions of the model are causal, meaning the model only takes information from previous data, and does not take into account future data in a given sequence, preserving the order of modelling the data. The predictions provided by the network are sequential, meaning after each sequence is predicted, it is fed back into the network to predict the next sample after that.

Optionally an ‘student’ feed-forward model can be added as seen in block 1105, rendering a trained ADCCNN at block 1104 to be the teaching model. This is similar to the Generative Adversarial Network, save for the difference being that the student network does not try to fool the teaching network like the generator does with the discriminator. Rather, the student network models the distribution of the ADCCNN, without necessarily producing one sample at a time, which enables the student to produce generations of pixels while operating under parallel processing, producing an output generation in real-time. This enables the present system to utilize both the learning strength of the ADCCNN, and the sampling of the student network, which is advised to be Inverse Autoregressive Flow (IFA). This distills probability distribution learned by the teaching network to the student network, that when deployed into production, can be thousands of times faster than the teaching network at producing the output. This means the result (when adding the student network) can generate from the first to last pixel altogether without generating one sample at a time in between, improving output resolution with number of pixels.

Whether tier I (a variation of RNN and GAN) is used, or tier II (a novel variation of CNNs with an additional student network learning the distribution in a manner that speeds up processing by enabling it to be computed in parallel), the output of either tier I or tier II is the produced video (and can be an image) in blocks 1107/1007

The third model is a video/image classification model that continuously scans images and videos generated from the second (generative) model and accurately tags what is inside them at block 1008. This is an image/video classifier which is known to, and can be constructed by, someone skilled in the art of building deep learning models of computer vision. CNNs, or DCNNs can be used here or a variation of one of these networks. Preferably, a pre-trained API 102 that is capable of recognizing, categorizing what is inside an image and annotating it with a description is utilized.

The third model in block 1008 serves the purpose of tagging and annotating all the output of the second (generative) model in order to create a searchable database through keywords of what the user was seeing. This would enable the user to swiftly search their own database to find specific things they saw rather than having to sift through all the videos (and images) generated over time.

The fourth modality at block 1009 is a ‘web-crawler’ zero-shot learning which enables the third model in block 1008 to learn by itself through usage without being explicitly trained on the newer classes by providing feedback from block 1010 to block 1008. Optional input can be provided to the network to assist the other components of the diagram in operating, such as the user's emotional state (in block 1013) derived from another capability 103. Another example is through covert brain-to-speech functionality, wherein the user could provide an input to the web-crawler from block 1013 to block 1009 in order to perform a function that uses the result of block 1007—for example, a user looking at a face of a celebrity can covertly say “System, who is this celebrity?”

The brain-to-speech component discussed below explains how this will be understood by the brain-to-speech and text capability which will trigger a command from block 1013 to block 1009 to perform a web search and return with a query response in block 1011 provided to the user through an interface which, by way of example shows a picture of the celebrity, their name, and a description of their bio, and for example, movies they have filmed.

By way of example, when being used after deployment, the user in this example is looking at a red panda, and the third model in block 1008 was not previously trained on recognizing a red panda. It provides an annotation to the web crawler as a description of the generated video that it's an animal that has reddish-brown fur, a long, shaggy tail, a waddling gait, white badges on the face. The fourth (web-crawler) modality in block 1009 uses this annotation to surf the web through a search engine such as Google, or a site such as Wikipedia and/or other sources of information and returns a response of probabilities that it is 90% likely to be a red-panda, and 10% likely to be a raccoon.

The fourth modality can also take input of the user's location through GPS, other location services, or any other inputs such as user preferences, social media information, other biosensors, as an additional feature to assist in its search, where for example red pandas are known to be found mostly in Southwestern China, and a location input of the user being in that region will indicate a higher likelihood of it being a red panda. This enables the third modality in block 1008 to learn by itself to categorize what is generated from the second model. It can be utilized by the user as a ‘Shazam for Vision’ meaning if there is a type of flower, animal, object or any other things that is animate or inanimate that the user is not familiar with, the user can, by looking at it, receive feedback from the result of third and fourth modality (blocks 1008, 1009, 1010) what the user is seeing.

The additional fourth modality can also be connected to another data source, for example, a database that has an image of every person and a description about them, recognize that person's face and provide through an interface to the user the person's bio, or their Wikipedia page, recognize if they were a celebrity and describe what movies they were in, as an example. The third and fourth modality can also by way of example, operate by recognizing an object from the video/image generated in block 1007, provide pricing of that object at other stores when the user is shopping in order for the user to know where that object is being sold and get the most competitive pricing, returned to the user through block 1011. The user can trigger a command to search for competitive pricing of an object through a button on an interface (which can also be triggered by way of mental command as will be described below), or by covertly providing a command from block 10013 to block 1009 such as “System, tell me what stores have a discount/competitive pricing on this TV?” These are examples to illustrate the API's usage and are not meant to limit the range of its applications 104.

Optionally, the probabilities can be returned to the user through an interface and the user is asked for input on whether the third and fourth model's classification of the physical characteristics seen in generated images/videos was correct. This would further improve self-learning of the third modality as the feedback loop shown between block 1010 and block 1008.

A weights Prediction calibration as shown in FIG. 9, and explained above may then be implemented.

Once the algorithm of four modalities (the first three being machine learning (e.g., deep learning) models that are trained, and the third model is attached to a fourth (web-crawling) modality and it is deployed within the API 102 along with its contextual requisite information such as its weights, it will be ready to receive new EEG signals 101 and generate in video form or in images what the user is looking at, a description of the user's vision, can be used as a method of identifying unknown animate/inanimate things, and as an on-command visual assistant to the user, being that the command is sent through another capability in as described in block 1013, or through a button available to the user on their user interface (which can also be triggered by way of mental commands as explained below).

When a new User B is wearing the/an EEG headset 12 they will calibrate with the same training data in the second training variant using the weights prediction calibration method described above. Once calibrated, the API 102 receives raw data derived from sensors that User B is wearing, and generate in video form (or can be image form) what the user is looking at, remembering, and imaging, in keywords (descriptions), as well as provide the functional value in block 1011 of the third model and additional modality described above.

The results of this capability 103 can be used as input into any one of the other capabilities, and/or in combination with them for an application 104.

### Decoding and Reconstructing What a User is Hearing

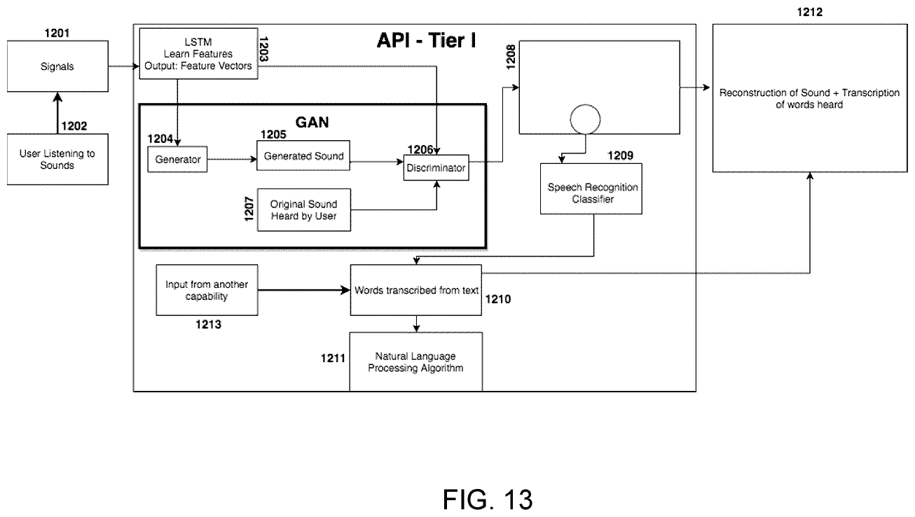

For this capability 103, signals 101 can be derived from the auditory cortex of the brain as seen in block 1201 of FIG. 13. Recommended electrode locations are, in this example:

A1, T9, T7, TP7, TP9, P9 and P7 for the left side of the brain; and

A2, T10, T8, CP8, CP10, P8, and P10 for the right side of the brain.

User A who, by way of example, is the user that will undergo the training process and whose data is used for training, undergoes an fMRI Scan and Digitization of head points. User A's fMRI scan and digitized head points are both co-Registered as seen in FIG. 6. Source localization is also performed, as seen in FIG. 7, but to areas responsible for processing auditory information, namely the entire auditory cortex on both sides of the brain.

There are two variant that will be described, for collecting a training dataset and which can be used to train the neural networks.

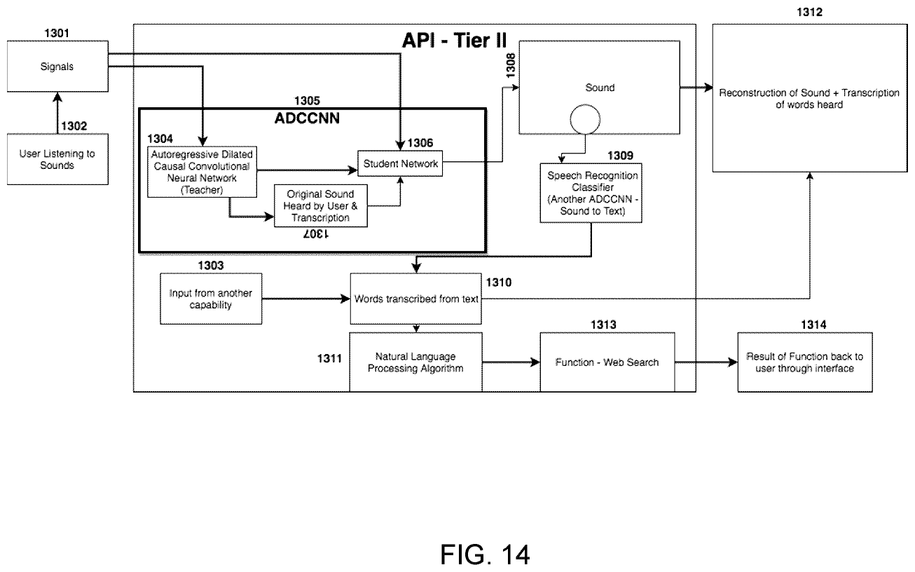

The first variant is to collect a dataset from a training user, User A, listening to target words in blocks 1202/1302 (see FIGS. 13 and 14). The training dataset along with the text of the word—for example the sound of the word “Hello” as an audio derivative along with the text “Hello” are fed as input into the algorithm of neural networks, to be trained.

The second variant is to collect a training dataset with User A listening to a categorized Phonology in blocks 1202/1302 (i.e. letters and phonemes that make up words). By way of example, “A, Ah, B, Beh” and their variants, done for every letter. Signals 101 are measured during training and labelled according to stimuli presented.

An algorithm which can be one of two tiers (tier I in FIG. 13, and tier II in FIG. 14) is constructed and an additional third model and fourth modality can be added as part of the algorithm, after one of the tiers is chosen.

The approach to constructing tier I can be characterized as follows, making reference to FIG. 13.

A neural network is constructed, namely by constructing an algorithm with two different models. The first model can be an LSTM Model as in block 1203, built for recognizing features. This model can be a hybrid of LSTM at the initial input layers to pick-up the features of time series with convolutional layers afterwards, or it can be another type of neural network (preferably a recurrent one) designed for picking up the features of time-series EEG data derived from the sensors. The first model is used to learn the features of EEG data surfacing on the cortical areas in response to sounds and produces an output of a feature vector that is used as input along with the original sound and transcription of what User A heard to the second model which is the GAN in FIG. 13.

The second model of tier I can be a VAE Convolutional Auto-Encoder, a variant of VAE, or a Generative Adversarial Network (GAN), Deconvolutional GAN, autoregressive Models, Stacked GAN, GAWNN, GAN-INT-CLAS, or a variant or substitute of any of the above. The second model takes the features as input and generates the sound that User A heard in audio form. Where the generator generates sound in block 1205 and the discriminator assesses how accurate the generated sound is relative to what it should be from the sounds heard at block 1202, the system can provide a feedback loop for the generative portion of the network to improve while the network is being trained.

Once the deep learning algorithm, utilizing two models (or more) designed to learn the features of raw EEG data derived from the cortical areas of the brain when a user listens to sounds and generating it, the algorithm is deployed within the API 102. The API 102 receives EEG signals 101 from block 1201 and generates a reconstruction of the sound in block 1208.

The second approach is implementing tier II in FIG. 14 can include the following.

First, this approach can include constructing an ADCCNN in block 1305 that directly obtains input from the raw signals in block 1301 and receives them in block 1304, which exhibits very large receptive fields to deal with long ranged temporal dynamics of input data needed to model the distribution of, and generate sound (or text) from brain-signals.

Each sample within an epoch/period of data is conditioned by the samples of all previous timestamps in that epoch and epochs before it. The convolutions of the model are causal, meaning the model only takes information from previous data, and does not take into account future data in a given sequence, preserving the order of modelling the data. The predictions provided by the network are sequential, meaning after each sequence is predicted, it is fed back into the network to predict the next sample after that. It is stacked with convolutional layers of a stride of one, which enables it to take input and produce output of the same dimensionality, perfect for modelling sequential data.

Optionally, a ‘student’ feed-forward model can be added as seen in block 1306, rendering a trained ADCCNN in block 1304 to be the teaching model. This is similar to the GAN, save for the difference being that the student network does not try to fool the teaching network like the generator does with discriminator, but rather the student network models the distribution of the ADCCNN, without necessarily producing one sample at a time. This enables the student to produce generations of text while operating under parallel processing, producing an output generation in real-time. This enables the present system to utilize both the learning strength of the ADCCNN, and the sampling of the student network, which is advised to be an IFA. This distills probability distribution learned by the teaching network to the student network, that when deployed into production is thousands of times faster than the teaching network at producing the output. This means the result (when adding the student network) will generate from the first to last audio sample altogether without generating one sample at a time in between.

Whether tier I (a variation of RNN and GAN) is used, or tier II (a novel variation of CNNs with an additional student network learning the distribution in a manner that speeds up processing by enabling it to be computed in parallel), the output of either tier I or tier II is the produced sound in block 1308 and block 1208. Afterwards, tier I or tier II (the ADCCNN with student network) can be used again in order to turn sound into text as a speech secognition classifier in blocks 1209 and 1309.

Weight prediction calibration as shown in FIG. 9 can be implemented where User B (a new user) listens to the same stimuli that was presented to User A during training, for a number of letters and their variants, then a prediction is made as to the weights of every other class and the final layer (or more) is fully replaced with newly predicted weights for User B—as with the process described above and seen in FIG. 9.

Optionally, the location of the sound heard by the user can be determined using the following process.

Firstly, User A can sit in a sound-isolated room wearing an EEG headset 12. A sound is presented to the user out loud at least four times—once from the north-western side of the room, once from the north-eastern side of the room, once from the south-western side of the room, once from the south-eastern side of the room. An exact distance is measured from where the user is sitting to the speaker, as well as the level of volume.

An LSTM Model receives raw signals 101 from the auditory cortices on the left and right sides of the brain and provides two different vectors of feature representations for a given sound, one from each side of the brain.

From signals 101 derived from the left-side of the brain, a feature vector (“FeatA”) is produced by the LSTM. For signals derived from the right-side of the brain, a feature vector (“FeatB”) is produced by the LSTM.

The difference between the FeatA and FeatB is calculated, being the delta.

A second model is constructed within the deep learning algorithm, which is a CNN that receives four inputs and is trained by classification (deep learning). The inputs into the CNN Model are: a delta difference between feature vectors produced by LSTM, a location of where the sound was produced (NW, NE, SW, SE), a level of volume of the speaker, the audio derivative of the sound itself, and an exact distance. It can be appreciated that the distance can be in meters, centimeters, or any measurement form, so long as it is used in consistently between all trials as the unit of distance measurement.

The CNN network is trained on measuring where the sound originated from (NW, NE, SW, or SE) by calculating the difference in values between FeatA and FeatB while taking into account the sound, location of sound, exact distance and volume of the sound.

This optional module, after training, can be deployed within the API 102 along with the sound generative model, enabling it to localize the source of the sound, in addition to generating it.

The results of this capability 103 can be used as input into any one of the other capabilities 103, and/or in combination with those capabilities 103, for an application 104.

### Decoding Mental Commands a User is Sending

For this capability 103, in a first variant (“Variant A” in this example), EEG signals 101 are derived from the motor cortical areas of the brain as with body movements. In a second variant (“Variant B” in this example), EEG signals 101 can be derived from all sensors available.

In Variant A, training data is collected from User A (the training user) as they move their body in accordance with what is shown in block 302 in FIG. 4. After replicating the training steps for decoding body movements in FIG. 4, the system has a trained deep learning model corresponding to block 303, namely “Model A”.

By way of example, the system can target six different commands to be given mentally to an application, although many more or fewer are possible. Example mental commands are Up, Down, Left, Right, Left-Click, On/Off.

After that, in Variant B, while taking into account the optimized weights of the network (Model A) from Variant A, User A imagines the mental commands while their signals 101 are being measured.

Here the system is implementing a weight-replacement calibration as per FIG. 8, on Model A from Variant A to Variant B. The reason being that performed body movements are more easily detected during training than imagined body movements. After doing a weight-replacement calibration on its final layer(s), while all previous layers are frozen using imagined body movements, enables the system to more accurately learn mental commands from raw data, and measure them in the future. The wording used herein, namely ‘imagined body movements’ should not be limiting, as this approach applies to any type of mental command given to an application 104.

The model (e.g., a deep learning model) in this case a hybrid of LSTM and CNN (but not limited to this choice) can quickly adapt to that user's brain as it has already learned the features of EEG through training of the first variant, retrained using the second variant in accordance with the number of classes of mental commands to be registered. The model is deployed within the API 102 and is ready to receive signals 101 and provide as a result, an accurate measurement of what mental command the user is giving.

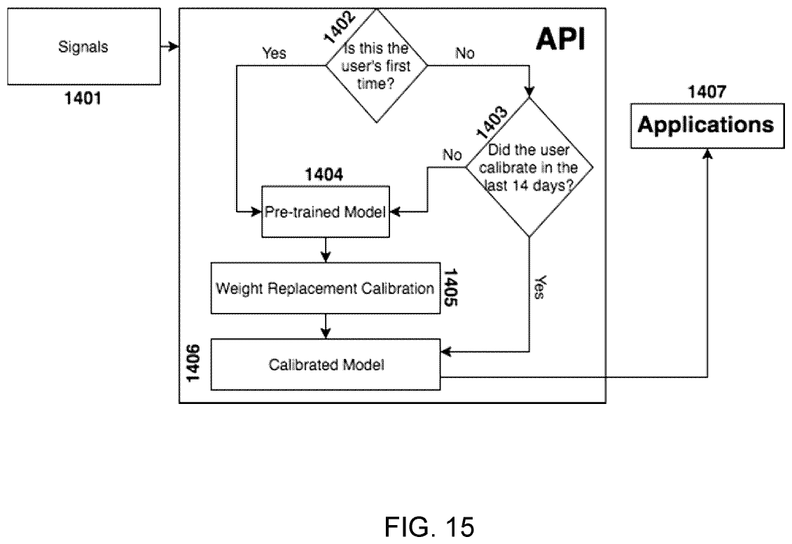

When new user, User B starts using the API 102, the system evaluates as shown in block 1402 of FIG. 15. If this is the user's first time the process goes to block 1404 which is the trained model from block 303 and performs a weight replacement calibration in block 1405. If this is not the user's first time, the system evaluates if the user has calibrated in the past fourteen days before that use, if the answer is not, it also goes back to block 1404 which is the model from 303, calibrates in block 1405 and becomes calibrated in block 1406. If the user did calibrate in the last fourteen days, the process proceeds directly to block 1406, rendering it ready to be used directly with any application 104 in block 1407, and in combination with another capability 103.

The results of this capability 103 can be used as input into any one of the other capabilities 103, and/or in combination with such capabilities 103, for an application 104.

### Brain-to-Text and Speech

This capability 103 enables the API 102 to produce, in text form and audio form, what the user was saying covertly, and/or overtly.

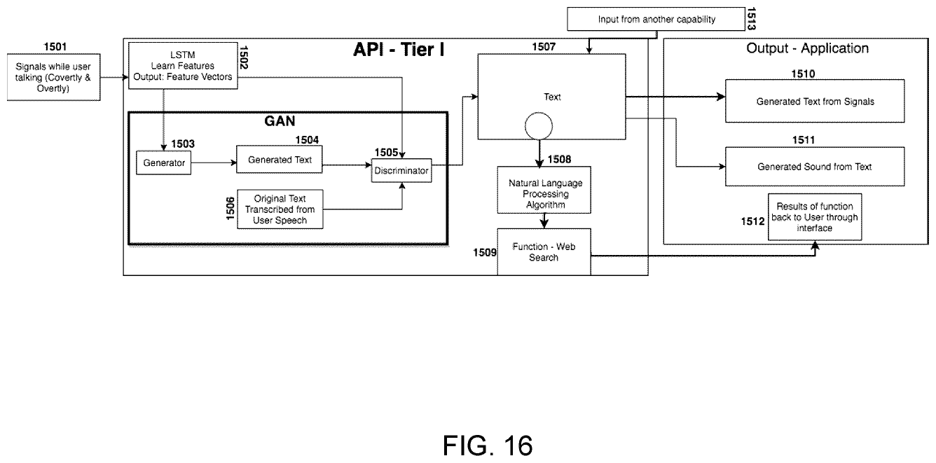

Signals 101 can be derived from the following electrode locations for block 1501 in FIG. 16:

F7, FT7, F5, FC5, F3, FC3, T7, C5, C3, C1, FC1, F1, TP7, CP5, CP3, CP1 and CPZ.

The electrode locations above are a recommendation, removing one more, or adding one or more other electrode locations will also work.

User A (the user who undertakes the training process), undergoes an fMRI and a digitization of head points, which are then both co-registered as seen in FIG. 6. Source localization can then be performed as per FIG. 7, but specifically to Broca's Area (speech production), Wernicke's Area (speech perception), motor cortex (speech articulation), and the ventral premotor cortex, and the entire cortex responsible for speech synthesis and perception.

Due to the principle of perceptual equivalence, there is an overlap of the neural substrates of when someone says a sentence out loud in block 1501, when they say it covertly, and when they hear it.

During the training process of this capability, the training data is collected as User A pronounces words out loud, and once the algorithm is deployed within the API 102, the next time User A covertly says a sentence through imagery (after calibrating), this sentence can be detected and generated in text form and/or audio.

There are two variants for training. In the first variant (“Variant A” in this example), User A is asked to pronounce target words. In the second training variant (“Variant B” in this example), User A is asked to pronounce categorized phonology, namely letters and phonemes that make up words—by way of example, “A, Ah, B, Beh” and their variants, done for every letter. Signals 101 are measured during training and labelled accordingly with the phonetic features.

An algorithm is constructed and can be one of two variable ways, and the difference between these novel and variable approaches will be discussed below.

Both variable approaches provide unique approaches to what has been observed traditionally. Traditionally, EEG signals 101 are filtered using known signal processing techniques like band-pass filtering, low-pass filtering and other techniques such as ICA or PCA, which are examples of these techniques. However, this implementation does not employ any of these techniques, and can be considered more effective through this implementation to construct and enable the deep learning algorithm to detect the desired signals rather than resorting to these traditional approaches. Traditional approaches include averaging the signals of each class to find what's known as the evoked response (the average signal for a specific class of body movement), or to find Event Related Potentials (ERP) like P300, isolating frequency bands by applying FFT or other wavelet transforms during intermediary signal processing, and then training an algorithm such as logistic regression or other ‘classical machine learning algorithms’.

This implementation does not perform intermediary signal processing, or average signals (which reduces the amount of data available for training the algorithm) since a neural network (as well as other deep learning models) requires a large amount of data for training. Instead, the system optimizes the network to find a shared pattern among all raw training examples provided to the network. Another example of learning the features can be to use two different models (or more) within the same algorithm rather than one.

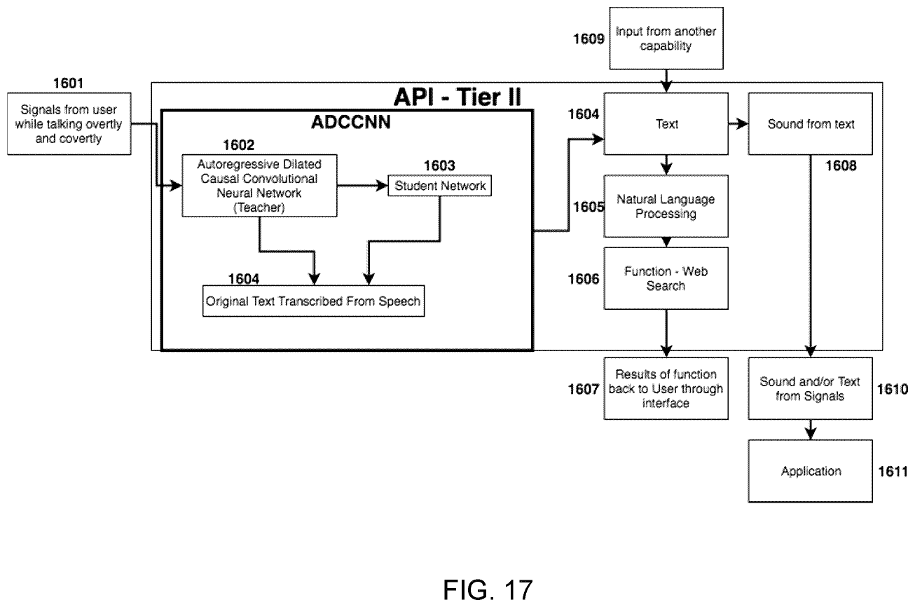

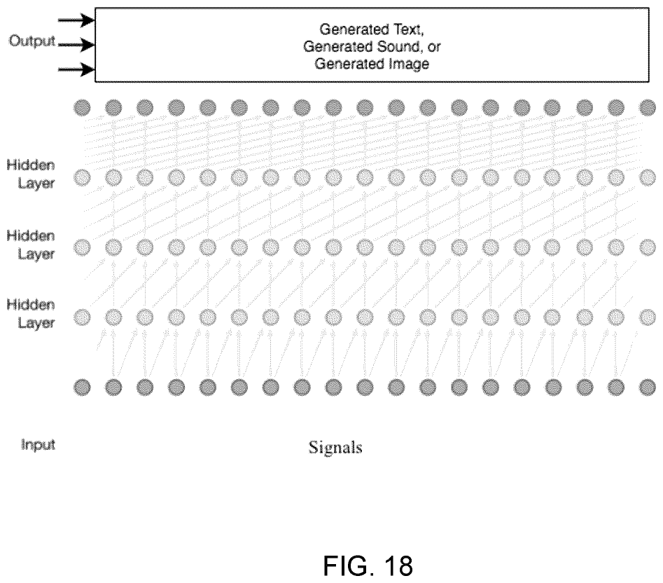

An algorithm which can be one of two tiers of models is illustrated in FIGS. 16 and 17 (tier I in FIG. 16, and tier II in FIG. 17). An additional modality can also be constructed and added to both tiers.

The following describes two approaches to constructing tier I. In a first approach, the system can construct a Model that's an LSTM in block 1502 that takes raw EEG signals 101 from the localized signals and provides as an output, a feature vector for every epoch/period of data. This can be an LSTM for every channel, an LSTM for all channels, or another type of Recurrent Neural Networks or variant of that.

The second model of tier I can be a VAE, Convolutional Auto-Encoders, or a GAN, Deconvolutional GANs, autoregressive models, stacked GAN, GAWNN, GAN-INT-CLS or a variant of any of the above, to generate an output from the input features of the first model. In this implementation by way of example, a GAN shown in FIG. 16, takes the feature vectors produced by the first model in block 1502 as input to the two sides of the GAN—the discriminator at block 1505 and the generator at block 1503. The generator generates text from the feature vectors of the sequence of brain signals 101 in that epoch in block 1504, and the discriminator assesses how accurate is the generated text in block 1504 in comparison to the original textual transcription of the sound produced overtly in block 1506. The discriminator then provides feedback through a loop to the generative portion of the network at block 1503 to improve while the network is being trained.

Once deployed, the second model of tier I generates in text form of what the user was saying (or imagined saying) when their EEG data was being recorded in block 1507.

The second approach is through implementing tier II in FIG. 17 as follows.

First, the second approach can include constructing a novel model based on ADCCNNs in block 1602, which exhibit very large receptive fields to deal with the long ranged temporal dynamics of input data needed to model the distribution of, and generate text or sound from brain-signals 101.

Each sample within an epoch/period of data is conditioned by the samples of all previous timestamps in that epoch and epochs before it. The convolutions of the model are causal, meaning the model only takes information from previous data, and does not take into account future data in a given sequence, preserving the order of modelling the data. The predictions provided by the network are sequential, meaning after each sequence is predicted, it is fed back into the network to predict the next sample after that. It is stacked with convolutional layers of a stride of one, which enables the system to take input and produce output of the same dimensionality, considered advantageous and ideal for modelling sequential data.

Optionally an ‘student’ feed-forward model can be added as seen in block 1603, rendering a trained ADCCNN in 1602 to be the teaching model. This is similar to the GAN, save for the difference being that the student network does not try to fool the teaching network like the generator does with the discriminator, but rather the student network models the distribution of the ADCCNN, without necessarily producing one sample at a time, which enables the student to produce generations of text while operating under parallel processing. As such, the system is commercially deployable to produce an output generation in real-time. This enables the system to utilize both the learning strength of the ADCCNN, and the sampling of the student network, which is advised to be an IFA. This distills probability distribution learned by the teaching network to the student network, that when deployed into production and can be thousands of times faster than the teaching network at producing the output. This means the result (when adding the student network) will generate from the first to last word altogether without generating one sample at a time in between.

Whether tier I (a variation of RNN and GAN) is used, or tier II (a novel variation of CNNs with an additional student network learning the distribution in a manner that speeds up processing by enabling it to be computed in parallel), the output of either tier I or tier II is the produced text in block 1507 and block 1604. Afterwards, tier I or tier II can be used again in order to turn text into speech in blocks 1510 and 1608. Alternatively, the original output can be speech, and tier I or tier II can be used to turn that speech into text. An ADCCNN is also used with a student network to generate sound from text.

Input can be provided from another capability 103 in blocks 1513 and 1609 or an external open data source. For example, emotions of the user from another one the capabilities 103 can be used as an input in order to provide an even more effective and natural tone to the produced speech in blocks 1511 and 1609.

The third model that can be employed is a Natural Language Processing (NLP) model that functions in two ways.

First, upon command by the user, the model can take the last thirty seconds of speech generated by the second model and run it against a database or web search in blocks 1509 and 1606 upon command either by the press of a button (which can be triggered by a mental command) or by naming covertly calling the System by a certain name. The result is returned in blocks 1509 and 1606 and shown in blocks 1512 and 1607 to the user.

Second, upon command from the user, the system can start listening to the upcoming covert speech. A user can covertly say “System, find me the nearest McDonalds”, and the result will be prompted through an interface in block 1607. The module in block 1605 triggers when, by way of example, the name “System” is covertly pronounced by the user, and after understanding what query/command/function the user is providing or requesting. It can do so in module 1606, and provide the results back to the user through an interface in block 1607 along with the sound and text generated by the second model. Together, these power any application 104, and in combination with any of the other capabilities 103.

The user can provide a command by saying a trigger phrase like “System”, which once recognized by block 1606 can utilize the result of another one or more of the capabilities 103. An example is covertly saying “System, what song am I listening to?” the sound is generated in block 1604, understood in block 1605, and a function in block 1606 queries against an external database or data source, e.g., Shazam's database, the sound that the user is listening to generated from the model in tier II or tier II, and provides the name of the song to the user in blocks 1607 or 1512. Another example command is for the user to ask “System, how was my mood today?” which would prompt 1606/1509 to query against emotions felt by the user in block 906 (see FIG. 10) throughout the day since the user woke up, and provide a result back to the user in 1607/1512 as to for example “You were happy 80% of the time, surprised 2% of the time, and angry 18% of the time”. This enables the user to better understand and quantify themselves. Another example is to covertly ask the system “What is my schedule for today?”, which would access the user's calendar (e.g., through Gmail or other application), and either show that to the user, or read out loud, acting as the user's personal assistant. That is, the system can be used to perform various functions and capabilities in an adaptable manner to assist the user. In another example, the user could ask the system to order food from a specific restaurant, which the system then finds the closest location and makes an order. Similarly, the user could ask the system what the weather would be like that day, or have the system let the user know if a particular contact sends a message (but otherwise suppresses all message notifications), etc.

Once deployed, a weight prediction calibration can be done by the user. The results of this capability 103 can be used as input into any one of the other capabilities 103, and/or in combination with such capabilities for an application 104.

It can be appreciated that the system can be deployed itself without the need for other tools. For example, in order to detect the user's emotions, the present disclosure enables understanding a user's emotional state solely from their brain signals 101, without the need for additional input. However, using a camera that detects for example whether someone smiles or frowns and provides additional input to the API 102 is possible, and such other inputs can be used to enhance a capability 103 or application 104 using one or more of the capabilities 103.

### Applications

### Dreams:

Due to the principle of perceptual equivalence, when for example a user looks at an object, imagines an object, or remembers how it looks, the same neurons are expected to activate. Therefore, generating a video of the user's vision when they are awake, enables generating a video of their dreams from their imagery during sleep.

A particularly central application of this technology is a dream recorder. A dream recorder requires measuring the user's perceptual experience, which is the combination of the capabilities 103 described above. A user wears a headset 12 when during sleep that generates signals 101 and provides them to the API 102 described above as input after the API 102 has been deployed and the user has calibrated the capabilities 103. The API 102 is a system that takes the signals 101 as input and provides back to the user the output of every capability 103. Therefore, a user wakes up and through a user interface which can be a web-application, phone application, a reconstruction in virtual reality or augmented reality, on TV or any other UI where the user can, for example: provide a mental command to a button on an interface through block 1407 to watch a video of their visual experience with a description of it as seen in blocks 1012 and 1112, hear a generated reconstruction of the sounds they heard while dreaming as seen in blocks 1212 and 1312 along with a transcription of the words heard, a generated reconstruction of the user's speech at blocks 1610, 1510 and 15111, as well as a description of their body activity resulting from blocks 405 to 406, which can also be represented by using an avatar of the user modelling the user's body activity (every body movement made) during the dream, as well as what emotions they felt all throughout the dream as shown in block 906.

The user can also search back in time through their dreams by using the virtual assistant described as “System” in by way of example saying “System, how many times have I dreamt of an elephant this week?” Where System would trigger block 1508 to query 1509 against blocks 1113 and 1108, returning the response to the user through an interface in block 1111.

The information from a person's dreams enables unprecedented frontiers in the capacity of quantified self, provides an empirical method of advancing the field of Oneirology providing it with significant credibility to reproduce research, and bridges the gap between spirit and science and a measurable form.

This allows the studying of advancement and discovery of human consciousness, or what will be termed “the collective consciousness” which is the perceptual experience of a group of individuals in certain geographical area whether small or across the globe.

The recording of dreams allows for various previously infeasible applications that use the results provided from dreams. These applications would not be possible without first building a dream recorder. Such as enabling therapists to diagnose patients in an unprecedented manner, by using their dreams which is one of the most discussed topics in Psychology by the leading Psychologists over centuries, such as Sigmund Freud and Carl Jung.

This would enable users to also understand their brain and perceptual experience during sleep, which is something every person on average spends 33% of their lives doing.

Another example of studying, advancing, or discovering new applications 104 within the collective consciousness is novel research experiments. For example, to see if in fact people do dream about things before they happen. Then, hypothetically speaking, correlating what a large group of people in a certain geographical area dream about with major events would provide a way of predicting the future. This is an example of novel research experiments that are only capable of being tested by using the above principles.

Another example is finding correlations between people that dream about each other. It can be appreciated that these are just examples to illustrate that there are myriad applications and research that can be implemented by using the system described herein, for the purposes of dreams which utilizes the combination of capabilities above. The combination of capabilities together, enabling the measurement of a user's perceptual experience can open the door to many possibilities and advancements across a wide range of industries as a result of being able to record dreams.

### Consciousness

The user's consciousness is their perceptual experience. The presently described system and capabilities thereof provides a way to measure consciousness of the user when they are awake, and when they are asleep.

### Medical

A range of applications in the medical sector are possible using the aforementioned system and capabilities thereof.

For example, patients with amputated limbs could use the capability of decoding body movements disclosed above in order to control a prosthetic limb in all degrees of freedom by thinking it, which prior to the aforementioned system's approach, was known to be limited to only continuous motion in certain directions. This enables free motions, meaning in any degree of movement that is not only limited to continuous motion of Upper Right, Upper Left, Lower Right, Lower Left, up, down, left, right, but in exact degrees of Upper Right, Upper Left, Lower Right, and Lower Left.

Using the capability aforementioned disclosure on providing mental commands, the patients can by way of example control a wheel chair by just their thoughts.

Using the perceptual experience by combining the output of all capabilities 103 can by way of example aid in assisting Alzheimer's patients by enabling them to re-experience their forgotten experiences and/or memories, and serve as a way of tracking their improvement progress.

### Locked-In Patients:

Locked-in patients such as patients with ALS can, be imagining moving their body control a user interface with their brain, enabling them to type words from a keyboard interface access the internet and entertainment such as playing games with the output of block 1406 replacing the keyboard/joystick/controller input in block 1407.

In addition to providing commands to a user interface using the approach in FIG. 15, locked-in patients are able to communicate with their loved ones and people around them through the capability 103 disclosed in FIGS. 16 and 17, which would allow them to communicate through brain to speech using the output of blocks 1510, 1511 and 1608 and 1610, with the emotional input of blocks 1513 and 1609 used to provide a tone to their generated voice. Patients can also type words through brain to speech using the capability 103 disclosed above and shown in FIGS. 16 and 17.

The capability of measuring a user's emotions is also used as a way of adapting applications 104 to their current preferences, as well as an expressive way letting know their loved ones, or those taking care of them such as a nurse, what emotions they are feeling.

Reports resulting from autonomous measurement of a patient's emotions can be used by doctors to further understand the mental state of their patient by seeing a report of their emotions over a day, or any certain period of time. This implementation can be used as a method of gauging mental improvement of the patient in the month of, for example November, versus the previous month of October. It can provide insight as to for example the person during November was on Average 80% of the time Happy/Joyful as opposed to in October when the patient was 40% of the time happy. Showing significant improvement in the method of diagnosing the mental state of locked-in patients and gauging improvement resulting from treatment if they were chronically depressed.

The combination of capabilities, together enable providing an unprecedented quality of life for Locked-in patients.

### Mind-Controlled, and Gesture Controlled Emotionally Adaptive Gaming (in Conventional Gaming as Well as Virtual Reality and Augmented Interfaces):

Using the combination of capabilities 103 described above, users are able to play a game using their body movement activity disclosed in FIG. 5, powering the application in block 406 or using mental commands powering applications in block 1407 as disclosed in FIG. 15. This replaces the need for keyboards/joysticks/controllers to be used. Where if the user is wearing a virtual reality or augmented reality headset, the signals 101 can be derived from sensors used in combination with the headpiece 12, or embedded in the headpiece 12.

If the user was playing a conventional game (what is meant by conventional here is one that is not VR/AR based, but rather one that by way of example was developed years ago and only uses a Playstation's joystick as input) the result of block 405 can be used as input into the game masked as the controller's input in block 406. This means that a specific bodily movement can send a command to the application in block 406 as if the user had pressed “X” button on a PlayStation Joystick.

The aforementioned disclosure enables users to not only play games in continuous motion, but goes steps further by enabling the modelling of free motion. The exact free motion of a user's body is modelled by the avatar in a game.

User Interfaces applicable here as a result of the capabilities disclosed in FIGS. 15 and 5 include menu navigation systems, volume control, or any type of interface that requires input from the user to control it through a keyboard/mouse/joystick/controller.

The applications mentioned here adapt to a user's emotions. For example, if a user is controlling a game such as Super Mario using block 406 or block 1407, the output of block 906 is used as input allowing the application to morph according to a user's experience. Hence for example if the user gets excited they get more bonus points in the game, or if the user feels stressed the difficulty of the game rises.

The combination of these Capabilities, together, provide an unprecedented approach to enabling mind/gesture controlled, emotionally adaptive gaming and user interfaces.

### Live Streaming a User's Vision (for Example at a Basketball Game Live):

In another example application 104, a user can by way of a mental command through block 1407 trigger a button to live-stream their perceptual experience using the aforementioned disclosure of capabilities 103. The user can do so using the generated visual experience with a description of it as seen in blocks 1012 and 1112, the generated reconstruction of the sounds they heard as seen in blocks 1212 and 1312 along with a transcription of the words heard, a generated reconstruction of the user's speech in blocks 1610, 1510 and 15111, as well as a description of their body activity resulting from blocks 405 to 406, which can also be represented by using an avatar of the user modelling the user's body activity (every body movement made), as well as what emotions they felt all throughout a period of time as shown in block 906.

This for example, replaces the need for using Snapchat, Periscope, etc. whereas the user can be wearing a headpiece 12 that sends signals to the API 102 of capabilities 103, live-streaming their perceptual experience, as they are experiencing it, without the need to use a phone to capture that experience. This is also much more entertaining for someone to watch, and it is different because it is through the actual point of view of the user that is doing the live-stream. This can be for example at a live basketball game where the user is sitting court-side, or a major event that is occurring at a point in time. The user can covertly or overtly say by way of example “System,” (or it can be that they choose any name to give their virtual assistant) “show me what I experienced 5 minutes ago”, and this would trigger block 1508 to query block 1509 against blocks 1113 and 1108, returning the response to the user through an interface in block 1111 (for vision), query against block 906 (for emotions), query against blocks 1513/1609 (for speech), query against blocks 1213 and 1303 (for auditory), and query against blocks 405 and 406 (for body movement modelling). These queries, through a user interface, would return the results of every one of those capabilities 103 over the desired period of time. The user could through a user interface provide a mental command from block 1407 to replay a certain perceptual experience.

All of the capabilities 103, together, form the perceptual experience of the user which enables the implementation of this application 104, by using the system described herein.

It may be noted that in this application 104, a user is able to go back and re-experience events, such as their daughter/son's graduation.

### Simulations, Military Training and Post-Traumatic Stress Disorder:

Simulations are being conducted in a number of ways—for example in military for training purposes simulate a battlefield experience for soldiers, in virtual reality therapy for overcoming a user's fear of heights, placing them in a virtual world where they are atop a roof looking down, and such exposure enables them to overcome their phobia/fear of heights. Measuring a user's perceptual experience while they undergo a simulation would render it much more effective when implemented.

For example, the doctor whose patient is undergoing simulation therapy is able to see exactly what their patient experiences as is generated from the combination of all the capabilities, by watching their perceptual experience. Is able to derive empirical reports on that experience as opposed to just the description provided by the user undergoing simulation.

Patients of post-traumatic stress disorder (which also include former military) remember episodes of previous events and also dream about them. Measuring their perceptual experience through the combination of all capabilities enables the doctors to better understand their condition, thus expose them to the most suitable form of simulation to help overcome those episodes and/or fears.

### Space Exploration:

In space exploration, astronauts are unable to carry equipment such as cameras into or outside the spaceship on a planet because high-powered electrics fail in space. In the case of EEG, the hardware is low-powered and compact rendering it usable in space. Reports by experiments conducted by NASA, the Canadian Space Agency (CSA), and other space agencies in conjunction with labs such as Harvard Medical School suggest that many astronauts take sleeping pills in space, and when they do, they are unable to go into deep sleep and report very bizarre dreams that only occur (are experienced) in space. The present system enables measuring an astronaut's perceptual experience for the purposes of studying why that special type of dreams only occurs in space.

When astronauts leave the ship, the perceptual experience of the astronaut (the combination of all capabilities 103 together derived from signals 101 generated by the headpiece 12 that the astronaut is wearing) can be stored and then sent back to their respective agencies in order to study the results of space exploration through the astronaut's point of view.

### Advertising—Measuring how People React to Commercials:

Significant efforts in advertising go towards understanding how consumers react to commercials, store design, pricing, packaging, new user interfaces, etc. The combination of capabilities 103 as disclosed above, enable unprecedented measurement of the user's perceptual experience towards, for example a new commercial or advertising campaign. This can be more accurate and efficient than using galvanic skin response, a camera for emotional facial recognition (because someone may be happy but not smile, or sad but not frown). This enables advertisers to get more value for every dollar they spend on figuring out how effective a commercial is to their target demographic and psychographic audience.

### Research (Lab):

A myriad of research applications 104 become possible as a result of being able to measure the user's perceptual experience. By way of example, schizophrenics imagine, see and experience things that others don't see. A schizophrenic patient is seen talking to themselves when in fact they describe that they are seeing people and or imaginary inanimate/animate things that doctors are unable to see. This causes a problem where schizophrenics are hard to diagnose and there is no way of understanding their experience in order to derive conclusive solutions.

The aforementioned disclosure, when the combination of all capabilities 103 is used, enables measuring the perceptual experience of a schizophrenic patient, hence the doctor is able to watch their patient's experience and see exactly what they report seeing, the sounds they imagine hearing, and is able to understand and diagnose their patient at a significantly higher level than before.

### Brain-Texting:

Users can, using the aforementioned disclosure in FIGS. 16 and 17 send a text by covertly speaking sentences and providing the results of blocks 1604 and 1510 directly and on command to applications 104 such as Whatsapp, Facebook Messenger, LinkedIn Messaging, etc. This can be done by, for example, saying covertly “System, send to Whatsapp contact ‘Omar’ the following message: stuck in traffic, I'll be there in 5 minutes.” Or “System send to my LinkedIn contact ‘Full Name’ the following: I look forward to our meeting.” This triggers blocks 1606/1509 to access Whatsapp, find the contact name and send the message. Or, in another example: “System take a snapshot of my point of view and send that to WhatsApp group ‘Ayyad Brothers’ which triggers blocks 1508/1605 to query 1509/1606 against 1013/1113 to use the result of 1112/1212 and send that to WhatsApp group through 1011/1111. The user's facial expressions as measured by block 405 by used as input through block 406 as input of what are known as Emojis.

This enables users to communicate through brain-to-text without the need to type or use audio-based commands overtly to their mobile phone. The user sends a text, on command, by speaking overtly to themselves.

### Pets (Dogs as an Example):

The aforementioned disclosure of capabilities 103 can be also used with pets, which taking dogs as an example, have evolved over years in the same social environment as humans, which means certain parts of the brain are similar such as vision (although dogs see things faster). The combination of capabilities 103 can be used as a way of monitoring one's pet in order to take better care of them. The capability of measure their dog's bodily activity when the owner is not at home, their emotional states, as well as what they hear and when they bark.

### Computer-to-Brain

This application 104 enables a user to ‘download’ information to their brain from a server hosting that information. For example, a user can download motor skills of a famous pianist, the motor skills of an all-star NBA player, or the perceptual experience of another user.

This is implemented by first using the perceptual experience measured from a first user, (User A who by way of example is a famous pianist). The motor cortical areas, as an example, are measured and decoded using the approach described above. The electrical signals along with their meaning (which is the output of the capability in block 406) are sent to a server which hosts that information.

User B, another user wears a device, which can be implantable such as a neural lace, implantable electrodes, or any other device that is capable of sending signals to the input of neurons such as transcranial magnetic stimulation TMS or transcranial direct current stimulation (TDCS), which stimulates a neuronal population with electrical signals.

The device worn by User B then stimulates the brain of the user by sending electrical signals to areas corresponding to what information is learned, for example, stimulating the motor cortical areas of the brain for User B sending electrical signals of User A while they were playing a song on the piano.

This approach can be used to enable for example, blind people to see, or deaf users to hear, where instead of User A, a camera sends video/pictures to the intermediary server which transforms pictures into electrical signals that are then sent to User B's brain to stimulate the visual cortices of that user.

Another example is to use a microphone to record sound which is sent to, and digitally transformed on an intermediary server to electrical signals, which then forwards that to a device that stimulates the brain of the user providing input to neurons in the auditory areas of the brain, enabling the deaf person to hear.

This also enables users to send information directly from one brain to another.

### Multi-User Lucid Dreaming:

This application 104 enables massive multi-user dream interactions, such as multiple users interacting in a virtual environment while lucidly dreaming, and this application 104 also enables customizing a user's dreams.

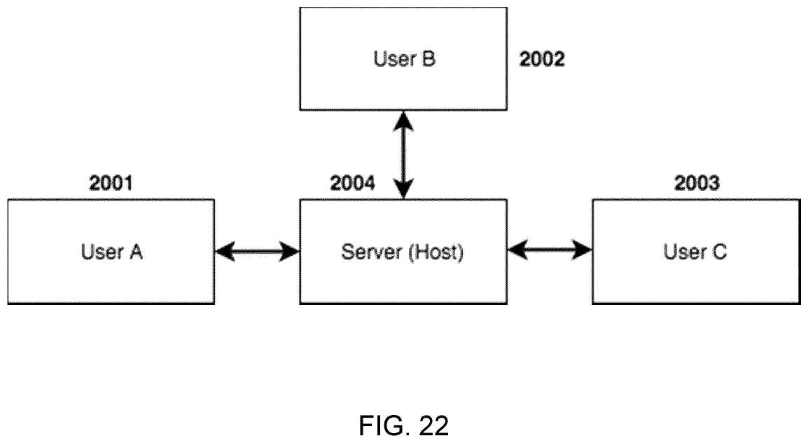

In this example, we assume multiple users as an example, User A, User B, and User C as shown in FIG. 22.

This application 104 includes providing stimuli to each user while they are asleep prompting them to realize that they are in a lucid dream. These stimuli can be delivered by stimulating (invoking) the brain of a user by sending electrical signals from the server to the device worn by the user (which stimulates the visual cortices of the brain) prompting them to see lights in their dream, which enables them to realize they are dreaming. Invoking the method can be using another approach such as stimulating the auditory areas of each user notifying them through sound that they are in a dream. The device worn by the user can be an implantable device such as tiny electrodes, a neural lace, or a non-invasive device such as TMS (Transcranial Magnetic Stimulation), or TCDS (Transcranial Direct Stimulation), or another worn device which is capable of sending electrical signals to the brain of the user.

Once a user realizes they are dreaming, they are capable of lucid dreaming, which is being aware that they are dreaming. When a user is aware they are dreaming, they are able to control that dream, as well as how they act in a dream.

A communication pathway between the User A and a server is established.

Meaning, the perceptual experience of User A who is dreaming (which is the output of each of the capabilities as disclosed above) is streamed to a server which hosts a virtual environment.

The server sends back to the User A, information by stimulating the corresponding regions of the brain of that user. This enables the server to receive the perceptual experience of each user, and send back information as to the virtual environment itself such as a shared scene with multiple users present in avatar form.

That communication pathway can be established between multiple users in a shared environment. This enables multiple users to be present in a virtual environment simultaneously whilst dreaming. Users can practice new skills (individually or together with other users), continue working on a project, or any other virtual experience for one or more users during a lucid dream. This may be described analogously as the internet of dreams—where people can be connected to each other during sleep, or otherwise connect individually, through a virtual environment for a virtual experience.

For simplicity and clarity of illustration, where considered appropriate, reference numerals may be repeated among the figures to indicate corresponding or analogous elements. In addition, numerous specific details are set forth in order to provide a thorough understanding of the examples described herein. However, it will be understood by those of ordinary skill in the art that the examples described herein may be practiced without these specific details. In other instances, well-known methods, procedures and components have not been described in detail so as not to obscure the examples described herein. Also, the description is not to be considered as limiting the scope of the examples described herein.

It will be appreciated that the examples and corresponding diagrams used herein are for illustrative purposes only. Different configurations and terminology can be used without departing from the principles expressed herein. For instance, components and modules can be added, deleted, modified, or arranged with differing connections without departing from these principles.

It will also be appreciated that any module or component exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the headset 12, module 14, cloud device 18, edge device 20, any component of or related thereto, etc., or accessible or connectable thereto. Any application or module herein described may be implemented using computer readable/executable instructions that may be stored or otherwise held by such computer readable media.

The steps or operations in the flow charts and diagrams described herein are just for example. There may be many variations to these steps or operations without departing from the principles discussed above. For instance, the steps may be performed in a differing order, or steps may be added, deleted, or modified.

Although the above principles have been described with reference to certain specific examples, various modifications thereof will be apparent to those skilled in the art as outlined in the appended claims.

1. A method for determining perceptual experiences, the method comprising:
obtaining a plurality of signals acquired by a measurement device comprising a plurality of sensors positioned to measure brain activity of users being measured by the measurement device;
providing the plurality of signals, without pre-processing, to a processing system comprising at least one deep learning module, the at least one deep learning module being configured to process the signals to generate at least one capability, wherein combinations of one or more of the at least one capability form the perceptual experiences; and
providing an output corresponding to a combination of one or more of the at least one capability to an application utilizing the corresponding perceptual experience.

2. The method of claim 1, further comprising training a machine learning algorithm in the deep learning module using signals measured during trials performed by a first user.

3. The method of claim 2, further comprising performing source localization in training the machine learning algorithm.

4. The method of claim 3, wherein the source localization comprises targeting areas of the brain according to the capability being generated.

5. The method of claim 2, wherein the machine learning algorithm comprises a convolution neural network (CNN).

6. The method of claim 5, wherein the CNN is trained using one of the following variants:
a) training a CNN model directly from raw signal data;
b) learning a feature representation of the signals through a plurality of different modules with a same algorithm; or
c) constructing an autoregressive dilated causal convolution neural network (ADCCNN) that directly receives the signals.

7. The method of claim 6, wherein in variant c), the ADCCNN is trained on providing an output of classes that indicates what functions were made by the user.

8. The method of claim 2, wherein the machine learning algorithm comprises a generative adversarial network.

9. The method of claim 2, further comprising conducting a calibration for a second user of the measurement device.

10. The method of claim 9, having the second user conduct the same trials as the first user.

11. The method of claim 10, wherein the calibration for the second user comprises using a same deep learning model with weights optimized to data derived from the first user, with at least one final layer of the network removed and replaced with a new layer optimized with weights associated with signals generated by the second user.

12. The method of claim 1, wherein the plurality of signals correspond to EEG signals acquired using a set of EEG sensors.

13. The method of claim 1, wherein the measurement device is a headset.

14. The method of claim 13, wherein the signals are acquired using the headset, and at least one of the processing system, the at least one capability, and the application is provided using a separate device.

15. The method of claim 14, wherein the separate device comprises an edge device coupled to the headset.

16. The method of claim 15, wherein the edge device communicates with a cloud device over a network to provide the at least one of the processing system, the at least one capability, and the application.

17. The method of claim 14, wherein the headset is configured to send at least signal data to a cloud device over a network.

18. The method of claim 1, wherein the at least one capability comprises measuring body movements.

19. The method of claim 18, wherein the deep learning module is trained by having the user trial a set of body movements.

20. The method of claim 18, wherein the body movements are modeled for continuous free motion to provide approximations of exact body movements of the user.

21. The method of claim 1, wherein the at least one capability comprises measuring a user's emotions.

22. The method of claim 21, wherein a plurality of emotions are determined according to a predefined categorization scheme, and measuring the emotions comprises eliciting emotions and measuring the brain activity to train the deep learning module to categorize emotions for that user.

23. The method of claim 22, wherein the deep learning module is constructed and trained on detecting the user's emotions using a pair of deep learning models, a recurrent neural network (RNN) as a first model that learns features from the signals and provides a feature vector as an input to a CNN as a second model that uses the feature vectors provided by the first model and further trains the deep learning module through classification.

24. The method of claim 23, wherein the RNN corresponds to a long-short-term-memory (LSTM) network.

25. The method of claim 21, wherein each of a plurality of emotions are output according to a scale.

26. The method of claim 25, further comprising combining a plurality of the emotions output according to the scale to identify a complex emotion.

27. The method of claim 1, wherein the at least one capability comprises decoding and reconstructing a user's vision.

28. The method of claim 27, wherein the decoding and reconstructing vision comprises: i) classifying vision training data using an RNN to learn features of the signal data in response to stimuli of images/videos, and ii) generating and classifying previously unseen images/videos in different categories, as well as the same category of images, as the stimuli of images.

29. The method of claim 1, wherein the at least one capability comprises decoding and reconstructing what a user is hearing.

30. The method of claim 29, wherein the decoding and reconstructing what a user is hearing comprises one of the following variants for collecting and training a dataset for the deep learning module:
a) collecting a dataset from a first user while the first user is listening to target words and feeding an audio derivative and text for the target word into an algorithm of neural networks; or
b) collecting a dataset with the first user listening to a categorized phonology and labeling signals according to stimuli presented along with textual transcriptions of sounds.

31. The method of claim 1, wherein the at least one capability comprises decoding mental commands from a user.

32. The method of claim 1, wherein the at least one capability comprises generating brain-to-text and/or speech.

33. The method of claim 1, wherein the application comprises a dream recorder that measures and records a user's perceptual experience during sleep.

34. The method of claim 33, wherein the dream recorder is operable to:
acquire the plurality of signals while the user is sleeping:
use the signals to generate an output corresponding to each of the capabilities;
generate the perceptual experience during sleep by combining the outputs for the capabilities; and
provide information indicative of the perceptual experience during sleep as a recording of the user's dream, through a user interface.

35. The method of claim 1, wherein the application comprises using the determined perceptual experience to measure the user's consciousness.

36. The method of claim 1, wherein the application comprises utilizing at least one of the capabilities in a medical application.

37. The method of claim 1, wherein the application comprises enabling locked-in patients to communicate according to the determined perceptual experience.

38. The method of claim 1, wherein the application comprises applying mind control or gesture controlled capabilities to one or more of: emotionally adaptive gaming, an augmented reality menu or interface, or a virtual reality menu or interface.

39. The method of claim 1, wherein the application comprises live streaming a user's vision.

40. The method of claim 1, wherein the application comprises measuring a user's perceptual experience during a simulation or training exercise.

41. The method of claim 1, wherein the application comprises remotely studying the user from a distance.

42. The method of claim 41, wherein the studying corresponds to astronauts.

43. The method of claim 1, wherein the application comprises measuring users' perceptual experience during consumer related activities for enhancing advertising.

44. The method of claim 1, wherein the application comprises measuring perceptual experiences for research.

45. The method of claim 1, wherein the application comprises brain texting.

46. The method of claim 1, wherein the application comprises monitoring a perceptual experience for a non-human subject.

47. The method of claim 46, wherein the non-human subject is a pet.

48. The method of claim 1, wherein the application comprises providing information to a user's brain from a computing device hosting the information.

49. The method of claim 1, wherein the application comprises multi-user dream interactions comprising a plurality of users connected to each other.

50. A computer readable medium comprising computer executable instructions for performing the method of claim 1.

51. A processing system for determining perceptual experiences, the system comprising at least one processor and at least one memory, the at least one memory storing computer executable instructions for performing the method of claim 1.
